(19) United States
(12) Patent Application Publication
(10) Pub. No.: US 2024/0091245 A1
(43) Pub. Date: Mar. 21, 2024

(54) PHARMACEUTICAL COMPOSITION, METHODS FOR TREATING AND USES THEREOF

(71) Applicant: Boehringer Ingelheim International GmbH, Ingelheim am Rhein (DE)

(72) Inventors: Uli Christian BROEDL, Mainz (DE); Odd-Erik JOHANSEN, Hoevik (NO); Eric Williams MAYOUX, Gambais (FR); Nima SOLEYMANLOU, Grand Island, NY (US); Maximilian von EYNATTEN, Erding (DE); Hans-Juergen WOERLE, Cully (CH); David Z.I. CHERNEY, Toronto (CA); Bruce A. PERKINS, Toronto (CA)

(21) Appl. No.: 18/515,359

(22) Filed: Nov. 21, 2023

Related U.S. Application Data
(63) Continuation of application No. 17/715,113, filed on Apr. 7, 2022, now abandoned.
Application No. 17/715,113 is a continuation of application No. 17/094,037, filed on Nov. 10, 2020, now Pat. No. 11,666,590.
Application No. 17/094,037 is a continuation of application No. 16/738,112, filed on Jan. 9, 2020, now abandoned.
Application No. 16/738,112 is a continuation of application No. 16/131,165, filed on Sep. 14, 2018, now abandoned.
Application No. 16/131,165 is a continuation of application No. 15/899,499, filed on Feb. 20, 2018, now abandoned.
Application No. 15/899,499 is a continuation of application No. 15/672,517, filed on Aug. 9, 2017, now abandoned.
Application No. 15/672,517 is a continuation of application No. 14/918,818, filed on Oct. 21, 2015, now abandoned.
Application No. 14/918,818 is a continuation of application No. 14/253,935, filed on Apr. 16, 2014, now abandoned.
(60) Provisional application No. 61/823,045, filed on May 14, 2013.
(60) Provisional application No. 61/813,223, filed on Apr. 18, 2013.

(51) Int. Cl. A61K 31/7048; A61K 9/20; A61K 9/28; A61K 31/155; A61K 31/522; A61K 38/28; A61K 45/06

(52) U.S. Cl. CPC A61K 31/7048; A61K 9/2018; A61K 9/2866; A61K 31/155; A61K 31/522; A61K 38/28; A61K 45/06

(57) ABSTRACT

The present invention relates to certain SGLT-2 inhibitors for treating, preventing, protecting against and/or delaying the progression of chronic kidney disease in patients, for example patients with prediabetes, type 1 or type 2 diabetes mellitus.

### TECHNICAL FIELD OF THE INVENTION

The present invention relates to certain SGLT-2 inhibitors for treating, preventing, protecting against and/or delaying the progression of chronic kidney disease in patients, for example patients with prediabetes, type 1 or type 2 diabetes mellitus.

### BACKGROUND OF THE INVENTION

Chronic kidney disease (CKD), also known as chronic renal disease, is a progressive loss in renal function over a period of months or years. The symptoms of worsening kidney function are non-specific, and chronic kidney disease is often diagnosed as a result of screening of people known to be at risk of kidney problems.

Chronic kidney disease may be identified by a blood test, for example for creatinine. Higher levels of creatinine indicate a lower glomerular filtration rate and as a result a decreased capability of the kidneys to excrete waste products.

CKD has been classified into 5 stages, where stage 1 is kidney damage with normal GFR (mL/min/1.73 m²) of ≥90; stage 2 is kidney damage with a mild decrease in GFR (GFR 60-89); stage 3 is a moderate decrease in GFR (GFR 30-59); stage 4 is a severe decrease in GFR (GFR 15-29); and stage 5 is kidney failure (GFR <15 or dialysis). Stage 5 CKD is often called End Stage Renal Disease (ESRD) and is synonymous with the now outdated terms chronic kidney failure (CKF) or chronic renal failure (CRF).

Albuminuria can also be a sign of kidney disease. Albuminuria has been classified into 3 categories, where category A1 reflects no albuminuria with albumin normal to mildly increased; category A2 which reflects microalbuminuria with albumin moderately increased; category A3 which reflects macroalbuminuria with albumin severely increased.

There is no specific treatment unequivocally shown to slow the worsening of chronic kidney disease and severe CKD requires renal replacement therapy, which may involve a form of dialysis, but ideally constitutes a kidney transplant.

Therefore, there is an unmet medical need for methods, medicaments and pharmaceutical compositions able to slow the worsening or progression of chronic kidney disease in patients, in particular patients at risk of renal diseases, for example patients with prediabetes, type 1 or type 2 diabetes mellitus.

### SUMMARY OF THE INVENTION

Accordingly, in one aspect, the present invention relates to certain SGLT-2 inhibitors, for example empagliflozin, for treating, preventing, protecting against, reducing the risk of, delaying the occurrence of and/or delaying the progression of chronic kidney disease in patients, for example patients with prediabetes, type 1 or type 2 diabetes mellitus.

In one embodiment, the present invention provides a method of treating, preventing, protecting against, reducing the risk of, delaying the occurrence of and/or delaying the progression of chronic kidney disease in a patient, said method comprising administering empagliflozin, optionally in combination with one or more other therapeutic substances, to the patient. In one aspect, the progression of said chronic kidney disease is the progression to end stage renal disease/kidney failure, or renal death in the patient.

In one embodiment, the present invention provides a method of treating, preventing, protecting against or delaying new onset of albuminuria in a patient, said method comprising administering empagliflozin, optionally in combination with one or more other therapeutic substances, to the patient. In one aspect, the patient is at risk for renal disease.

In one aspect, in one of the above methods, the patient is a patient with prediabetes, type 1 or type 2 diabetes mellitus. In one aspect, the patient has or is at risk of a cardiovascular disease. In one aspect, the patient is a patient with prediabetes, type 1 or type 2 diabetes mellitus and with or at risk of a cardiovascular disease.

In one embodiment, the present invention provides a method of treating, preventing, protecting against or delaying the progression from no albuminuria to micro- or macroalbuminuria in a patient at risk for renal disease, said method comprising administering empagliflozin, optionally in combination with one or more other therapeutic substances, to the patient.

In one embodiment, the present invention provides a method of treating, preventing, protecting against or delaying the progression from microalbuminuria to macroalbuminuria in a patient with chronic kidney disease, said method comprising administering empagliflozin, optionally in combination with one or more other therapeutic substances, to the patient.

In one aspect, in a method above the patient is a patient with prediabetes, type 1 or type 2 diabetes mellitus.

In one embodiment, the present invention provides a method for treating, preventing, protecting against or delaying the progression of chronic kidney disease in a patient, in particular a patient with chronic kidney disease, said method administering empagliflozin, optionally in combination with one or more other therapeutic substances, to the patient. In one aspect, the method is for preventing, protecting against or delaying loss of eGFR, for example sustained eGFR loss of >50%, in said patient. In one aspect, the patient is a patient with prediabetes, type 1 or type 2 diabetes mellitus.

In one embodiment, the present invention provides a method of treating, preventing, protecting against or delaying the occurrence of:
new onset of albuminuria,
doubling of serum creatinine level accompanied by an eGFR (based on modification of diet in renal disease (MDRD) formula) ≤45 mL/min/1.73 m²,
need for continuous renal replacement therapy, or
death due to renal disease,
   in a patient, said method comprising administering empagliflozin, optionally in combination with one or more other therapeutic substances, to the patient.

In one aspect, the patient has chronic kidney disease. In one aspect, the patient is a patient with prediabetes, type 1 or type 2 diabetes mellitus. In one aspect, the patient has or is at risk of a cardiovascular disease. In one aspect, the patient is a patient with prediabetes, type 1 or type 2 diabetes and with or at risk of a cardiovascular disease. In one aspect, the patient has chronic kidney disease and is a patient with prediabetes, type 1 or type 2 diabetes mellitus.

In one aspect, in any one of the methods above the patient is a patient with one or more cardiovascular risk factors selected from A), B), C) and D), for example a patient with type 1 or type 2 diabetes mellitus or with pre-diabetes with one or more cardiovascular risk factors selected from A), B), C) and D):

A) previous or existing vascular disease selected from myocardial infarction, coronary artery disease, percutaneous coronary intervention, coronary artery by-pass grafting, ischemic or hemorrhagic stroke, congestive heart failure, and peripheral occlusive arterial disease,

B) advanced age >/=60-70 years, and

C) one or more cardiovascular risk factors selected from
advanced type 1 or type 2 diabetes mellitus >10 years duration,
hypertension,
current daily cigarette smoking,
dyslipidemia,
obesity,
age >/=40,
metabolic syndrome, hyperinsulinemia or insulin resistance, and
hyperuricemia, erectile dysfunction, polycystic ovary syndrome, sleep apnea, or family history of vascular disease or cardiomyopathy in first-degree relative;

D) one or more of the following:
confirmed history of myocardial infarction,
unstable angina with documented multivessel coronary disease or positive stress test,
multivessel Percutaneous Coronary Intervention,
multivessel Coronary Artery By-pass Grafting (CABG),
history of ischemic or hemorrhagic stroke,
peripheral occlusive arterial disease.

In one aspect of the present invention, a patient having cardiovascular disease is defined as having at least one of the following:
Confirmed history of myocardial infarction; or
Evidence of multivessel coronary artery disease, in 2 or more major coronary arteries, irrespective of the revascularization status, i.e.
   a) Either the presence of a significant stenosis (imaging evidence of at least 50% narrowing of the luminal diameter measured during a coronary angiography or a multi-sliced computed tomography angiography), in 2 or more major coronary arteries,
   b) Or a previous revascularisation (percutaneous transluminal coronary angioplasty with or without stent, or coronary artery bypass grafting), in 2 or more major coronary arteries,
   c) Or the combination of previous revascularisation in one major coronary artery (percutaneous transluminal coronary angioplasty with or without stent, or coronary artery bypass grafting), and the presence of a significant stenosis in another major coronary artery (imaging evidence of at least 50% narrowing of the luminal diameter measured during a coronary angiography or a multi-sliced computed tomography angiography),
      Note: A disease affecting the left main coronary artery is considered as a 2-vessel disease.
Evidence of a single vessel coronary artery disease with:
   a) The presence of a significant stenosis i.e. the imaging evidence of at least 50% narrowing of the luminal diameter of one major coronary artery in patients not subsequently successfully revascularised (measured during a coronary angiography or a multi-sliced computed tomography angiography)
   b) And at least one of the following (either (i) or (ii)):
      i. A positive non invasive stress test, confirmed by either:
         1. A positive exercise tolerance test in patients without a complete left bundle branch block, Wolff-Parkinson-White syndrome, or paced ventricular rhythm, or
         2. A positive stress echocardiography showing regional systolic wall motion abnormalities, or
         3. A positive scintigraphic test showing stress-induced ischemia, i.e. the development of transient perfusion defects during myocardial perfusion imaging;
      ii. Or patient discharged from hospital with a documented diagnosis of unstable angina within 12 months prior to selection.
Episode of unstable angina with confirmed evidence of coronary multivessel or single vessel disease as defined above.
History of ischemic or haemorrhagic stroke
Presence of peripheral artery disease (symptomatic or not) documented by either: previous limb angioplasty, stenting or bypass surgery; or previous limb or foot amputation due to circulatory insufficiency; or angiographic evidence of significant (>50%) peripheral artery stenosis in at least one limb; or evidence from a non-invasive measurement of significant (>50% or as reported as hemodynamically significant) peripheral artery stenosis in at least one limb; or ankle brachial index of <0.9 in at least one limb.

In one aspect of the present invention, a patient having cardiovascular disease is defined as having at least one of the following:
a) Confirmed history of myocardial infarction,
b) Unstable angina with documented multivessel coronary disease (at least two major coronary arteries in angiogram) or positive stress test (ST segment depression >=2 mm or a positive nuclear perfusion scintigram),
c) Multivessel Percutaneous Coronary Intervention (PCI),
d) Multivessel Coronary Artery By-pass Grafting (CABG), including with recurrent angina following surgery,
e) History of ischemic or hemorrhagic stroke,
f) Peripheral occlusive arterial disease (previous limb bypass surgery or percutaneous transluminal angioplasty; previous limb or foot amputation due to circulatory insufficiency, angiographic or imaging detected (for example: ultrasound, Magnetic Resonance Imaging) significant vessel stenosis of major limb arteries).

In one aspect, in any one of the methods above the one or more other therapeutic substances are selected from other antidiabetic substances.

In one aspect, any one of the methods above comprises administering empagliflozin in combination with metformin, with linagliptin or with metformin and linagliptin.

In one aspect, in any one of the methods above the one or more other therapeutic substances is a RAAS inhibitor. In one aspect, the one or more other therapeutic substances is a direct renin inhibitor, an Angiotensin-Converting Enzyme (ACE) inhibitor and/or an angiotensin II receptor blocker (ARB).

In one aspect, any one of the methods above comprises administering empagliflozin in combination with a RAAS inhibitor. In one aspect, any one of the methods above comprises administering empagliflozin in combination with a direct renin inhibitor, an Angiotensin-Converting Enzyme (ACE) inhibitor and/or an angiotensin II receptor blocker (ARB).

In one aspect, in any one of the methods above empagliflozin is administered orally in a total daily amount of 10 mg or 25 mg. In one aspect empagliflozin is administered as a pharmaceutical composition comprising 10 mg or 25 mg of empagliflozin.

In one aspect of the present invention, in a method or use disclosed herein a patient is a patient with type 2 diabetes mellitus (or type 2 diabetes patient), a patient treated for type 2 diabetes mellitus, a patient diagnosed with type 2 diabetes mellitus or a patient in need of treatment for type 2 diabetes mellitus. In one aspect, a patient is a patient with pre-diabetes.

In one aspect of the present invention, in a method or use disclosed herein a patient is a patient with obesity-related Glomerulopathy, a patient with perihilar fokal-segmental glomerulosclerosis or a patient with IgA nephropathy.

Accordingly, in one embodiment, the present invention provides a method of treating, preventing, protecting against, reducing the risk of, delaying the occurrence of and/or delaying the progression of chronic kidney disease in a patient with obesity-related Glomerulopathy, in a patient with perihilar fokal-segmental glomerulosclerosis or in a patient with IgA nephropathy.

The present invention further provides for empagliflozin or a pharmaceutical composition comprising empagliflozin for use as a medicament in any one of the methods described herein.

The present invention further provides for empagliflozin in combination with one or more other therapeutic substances, for example selected from other antidiabetic substances, in particular metformin, linagliptin or metformin and linagliptin, or a pharmaceutical composition comprising empagliflozin and one or more other therapeutic substances, for example selected from other antidiabetic substances, in particular metformin, linagliptin or metformin and linagliptin, for use as a medicament in any one of the methods described herein.

The present invention further provides for empagliflozin in combination with a RAAS inhibitor, in particular a direct renin inhibitor, an Angiotensin-Converting Enzyme (ACE) inhibitor and/or an angiotensin II receptor blocker (ARB), or a pharmaceutical composition comprising empagliflozin and a RAAS inhibitor, in particular a direct renin inhibitor, an Angiotensin-Converting Enzyme (ACE) inhibitor and/or an angiotensin II receptor blocker (ARB), for use as a medicament in any one of the methods described herein.

The present invention further provides for empagliflozin or a pharmaceutical composition comprising empagliflozin for use in the treatment of any one of the diseases or conditions described herein.

The present invention further provides for empagliflozin in combination with one or more other therapeutic substances, for example selected from other antidiabetic substances, in particular metformin, linagliptin or metformin and linagliptin, or a pharmaceutical composition comprising empagliflozin and one or more other therapeutic substances, for example selected from other antidiabetic substances, in particular metformin, linagliptin or metformin and linagliptin, for use in the treatment of any one of the diseases or conditions described herein.

The present invention further provides for empagliflozin in combination with a RAAS inhibitor, in particular a direct renin inhibitor, an Angiotensin-Converting Enzyme (ACE) inhibitor and/or an angiotensin II receptor blocker (ARB), or a pharmaceutical composition comprising empagliflozin and a RAAS inhibitor, in particular a direct renin inhibitor, an Angiotensin-Converting Enzyme (ACE) inhibitor and/or an angiotensin II receptor blocker (ARB), for use in the treatment of any one of the diseases or conditions described herein.

The present invention further provides for empagliflozin or a pharmaceutical composition comprising empagliflozin for use in the manufacture of a medicament for use in any one of the methods described herein.

The present invention further provides for empagliflozin in combination with one or more other therapeutic substances, for example selected from other antidiabetic substances, in particular metformin, linagliptin or metformin and linagliptin, or a pharmaceutical composition comprising empagliflozin and one or more other therapeutic substances, for example selected from other antidiabetic substances, in particular metformin, linagliptin or metformin and linagliptin, for use in the manufacture of a medicament for use in any one of the methods described herein.

The present invention further provides for empagliflozin in combination with a RAAS inhibitor, in particular a direct renin inhibitor, an Angiotensin-Converting Enzyme (ACE) inhibitor and/or an angiotensin II receptor blocker (ARB), or a pharmaceutical composition comprising empagliflozin and a RAAS inhibitor, in particular a direct renin inhibitor, an Angiotensin-Converting Enzyme (ACE) inhibitor and/or an angiotensin II receptor blocker (ARB), for use in the manufacture of a medicament for use in any one of the methods described herein.

### Definitions

The term “active ingredient” of a pharmaceutical composition according to the present invention means the SGLT2 inhibitor according to the present invention. An “active ingredient” is also sometimes referred to herein as an “active substance”.

The term “body mass index” or “BMI” of a human patient is defined as the weight in kilograms divided by the square of the height in meters, such that BMI has units of kg/m².

The term “overweight” is defined as the condition wherein the individual has a BMI greater than or 25 kg/m² and less than 30 kg/m². The terms “overweight” and “pre-obese” are used interchangeably.

The terms “obesity” or “being obese” and the like are defined as the condition wherein the individual has a BMI equal to or greater than 30 kg/m². According to a WHO definition the term obesity may be categorized as follows: the term “class I obesity” is the condition wherein the BMI is equal to or greater than 30 kg/m² but lower than 35 kg/m²; the term “class II obesity” is the condition wherein the BMI is equal to or greater than 35 kg/m² but lower than 40 kg/m²; the term “class III obesity” is the condition wherein the BMI is equal to or greater than 40 kg/m².

The indication obesity includes in particular exogenic obesity, hyperinsulinaemic obesity, hyperplasmic obesity, hyperphyseal adiposity, hypoplasmic obesity, hypothyroid obesity, hypothalamic obesity, symptomatic obesity, infantile obesity, upper body obesity, alimentary obesity, hypogonadal obesity, central obesity, visceral obesity, abdominal obesity.

The term “visceral obesity” is defined as the condition wherein a waist-to-hip ratio of greater than or equal to 1.0 in men and 0.8 in women is measured. It defines the risk for insulin resistance and the development of pre-diabetes.

The term “abdominal obesity” is usually defined as the condition wherein the waist circumference is >40 inches or 102 cm in men, and is >35 inches or 94 cm in women. With regard to a Japanese ethnicity or Japanese patients abdominal obesity may be defined as waist circumference >85 cm in men and >90 cm in women (see e.g. investigating committee for the diagnosis of metabolic syndrome in Japan).

The term “euglycemia” is defined as the condition in which a subject has a fasting blood glucose concentration within the normal range, greater than 70 mg/dl (3.89 mmol/L) and less than 100 mg/dl (5.6 mmol/L). The word “fasting” has the usual meaning as a medical term.

The term “hyperglycemia” is defined as the condition in which a subject has a fasting blood glucose concentration above the normal range, greater than 100 mg/dl (5.6 mmol/L). The word “fasting” has the usual meaning as a medical term.

The term “hypoglycemia” is defined as the condition in which a subject has a blood glucose concentration below the normal range, in particular below 70 mg/dl (3.89 mmol/L).

The term “postprandial hyperglycemia” is defined as the condition in which a subject has a 2 hour postprandial blood glucose or serum glucose concentration greater than 200 mg/dL (11.11 mmol/L).

The term “impaired fasting blood glucose” or “IFG” is defined as the condition in which a subject has a fasting blood glucose concentration or fasting serum glucose concentration in a range from 100 to 125 mg/dl (i.e. from 5.6 to 6.9 mmol/l), in particular greater than 110 mg/dL and less than 126 mg/dl (7.00 mmol/L). A subject with “normal fasting glucose” has a fasting glucose concentration smaller than 100 mg/dl, i.e. smaller than 5.6 mmol/l.

The term “impaired glucose tolerance” or “IGT” is defined as the condition in which a subject has a 2 hour postprandial blood glucose or serum glucose concentration greater than 140 mg/dl (7.78 mmol/L) and less than 200 mg/dl (11.11 mmol/L). The abnormal glucose tolerance, i.e. the 2 hour postprandial blood glucose or serum glucose concentration can be measured as the blood sugar level in mg of glucose per dL of plasma 2 hours after taking 75 g of glucose after a fast. A subject with “normal glucose tolerance” has a 2 hour postprandial blood glucose or serum glucose concentration smaller than 140 mg/dl (7.78 mmol/L).

The term “hyperinsulinemia” is defined as the condition in which a subject with insulin resistance, with or without euglycemia, has fasting or postprandial serum or plasma insulin concentration elevated above that of normal, lean individuals without insulin resistance, having a waist-to-hip ratio <1.0 (for men) or <0.8 (for women).

The terms “insulin-sensitizing”, “insulin resistance-improving” or “insulin resistance-lowering” are synonymous and used interchangeably.

The term “insulin resistance” is defined as a state in which circulating insulin levels in excess of the normal response to a glucose load are required to maintain the euglycemic state (Ford E S, et al. JAMA. (2002) 287: 356-9). A method of determining insulin resistance is the euglycaemic-hyperinsulinaemic clamp test. The ratio of insulin to glucose is determined within the scope of a combined insulin-glucose infusion technique. There is found to be insulin resistance if the glucose absorption is below the 25th percentile of the background population investigated (WHO definition). Rather less laborious than the clamp test are so called minimal models in which, during an intravenous glucose tolerance test, the insulin and glucose concentrations in the blood are measured at fixed time intervals and from these the insulin resistance is calculated. With this method, it is not possible to distinguish between hepatic and peripheral insulin resistance.

Furthermore, insulin resistance, the response of a patient with insulin resistance to therapy, insulin sensitivity and hyperinsulinemia may be quantified by assessing the “homeostasis model assessment to insulin resistance (HOMA-IR)” score, a reliable indicator of insulin resistance (Katsuki A, et al. Diabetes Care 2001; 24: 362-5). Further reference is made to methods for the determination of the HOMA-index for insulin sensitivity (Matthews et al., Diabetologia 1985, 28: 412-19), of the ratio of intact proinsulin to insulin (Forst et al., Diabetes 2003, 52(Suppl.1): A459) and to an euglycemic clamp study. In addition, plasma adiponectin levels can be monitored as a potential surrogate of insulin sensitivity. The estimate of insulin resistance by the homeostasis assessment model (HOMA)-IR score is calculated with the formula (Galvin P, et al. Diabet Med 1992; 9: 921-8):

HOMA-IR =[fasting serum insulin (μU/mL)]×[fasting plasma glucose(mmol/L)/22.5]

Insulin resistance can be confirmed in these individuals by calculating the HOMA-IR score. For the purpose of this invention, insulin resistance is defined as the clinical condition in which an individual has a HOMA-IR score >4.0 or a HOMA-IR score above the upper limit of normal as defined for the laboratory performing the glucose and insulin assays.

As a rule, other parameters are used in everyday clinical practice to assess insulin resistance. Preferably, the patient's triglyceride concentration is used, for example, as increased triglyceride levels correlate significantly with the presence of insulin resistance.

Individuals likely to have insulin resistance are those who have two or more of the following attributes: 1) overweight or obese, 2) high blood pressure, 3) hyperlipidemia, 4) one or more 1^{st} degree relative with a diagnosis of IGT or IFG or type 2 diabetes.

Patients with a predisposition for the development of IGT or IFG or type 2 diabetes are those having euglycemia with hyperinsulinemia and are by definition, insulin resistant. A typical patient with insulin resistance is usually overweight or obese. If insulin resistance can be detected, this is a particularly strong indication of the presence of pre-diabetes. Thus, it may be that in order to maintain glucose homoeostasis a person needs 2-3 times as much insulin as a healthy person, without this resulting in any clinical symptoms.

“Pre-diabetes” is a general term that refers to an intermediate stage between normal glucose tolerance (NGT) and overt type 2 diabetes mellitus (T2DM), also referred to as intermediate hyperglycaemia. As such, it represents 3 groups of individuals, those with impaired glucose tolerance (IGT) alone, those with impaired fasting glucose (IFG) alone or those with both IGT and IFG. IGT and IFG usually have distinct pathophysiologic etiologies, however also a mixed condition with features of both can exist in patients. Therefore in the context of the present invention a patient being diagnosed of having “pre-diabetes” is an individual with diagnosed IGT or diagnosed IFG or diagnosed with both IGT and IFG. Following the definition according to the American Diabetes Association (ADA) and in the context of the present invention a patient being diagnosed of having “pre-diabetes” is an individual with:

a) a fasting plasma glucose (FPG) concentration <100 mg/dL [1 mg/dL=0.05555 mmol/L] and a 2-hour plasma glucose (PG) concentration, measured by a 75-g oral glucose tolerance test (OGTT), ranging between ≥140 mg/dl and <200 mg/dL (i.e., IGT); or

b) a fasting plasma glucose (FPG) concentration between ≥100 mg/dl and <126 mg/dl and a 2-hour plasma glucose (PG) concentration, measured by a 75-g oral glucose tolerance test (OGTT) of <140 mg/dl (i.e., IFG); or

c) a fasting plasma glucose (FPG) concentration between ≥100 mg/dl and <126 mg/dl and a 2-hour plasma glucose (PG) concentration, measured by a 75-g oral glucose tolerance test (OGTT), ranging between ≥140 mg/dl and <200 mg/dL (i.e., both IGT and IFG).

Patients with “pre-diabetes” are individuals being pre-disposed to the development of type 2 diabetes. Pre-diabetes extends the definition of IGT to include individuals with a fasting blood glucose within the high normal range ≥100 mg/dl (J. B. Meigs, et al. Diabetes 2003; 52: 1475-1484). The scientific and medical basis for identifying pre-diabetes as a serious health threat is laid out in a Position Statement entitled “The Prevention or Delay of Type 2 Diabetes” issued jointly by the American Diabetes Association and the National Institute of Diabetes and Digestive and Kidney Diseases (Diabetes Care 2002; 25: 742-749).

The methods to investigate the function of pancreatic beta-cells are similar to the above methods with regard to insulin sensitivity, hyperinsulinemia or insulin resistance: An improvement of beta-cell function can be measured for example by determining a HOMA-index for beta-cell function (Matthews et al., Diabetologia 1985, 28: 412-19), the ratio of intact proinsulin to insulin (Forst et al., Diabetes 2003, 52(Suppl.1): A459), the insulin/C-peptide secretion after an oral glucose tolerance test or a meal tolerance test, or by employing a hyperglycemic clamp study and/or minimal modeling after a frequently sampled intravenous glucose tolerance test (Stumvoll et al., Eur J Clin Invest 2001, 31: 380-81).

The term “type 1 diabetes” is defined as the condition in which a subject has, in the presence of autoimmunity towards the pancreatic beta-cell or insulin, a fasting blood glucose or serum glucose concentration greater than 125 mg/dl (6.94 mmol/L). If a glucose tolerance test is carried out, the blood sugar level of a diabetic will be in excess of 200 mg of glucose per dL (11.1 mmol/l) of plasma 2 hours after 75 g of glucose have been taken on an empty stomach, in the presence of autoimmunity towards the pancreatic beta cell or insulin. In a glucose tolerance test 75 g of glucose are administered orally to the patient being tested after 10-12 hours of fasting and the blood sugar level is recorded immediately before taking the glucose and 1 and 2 hours after taking it. The presence of autoimmunity towards the pancreatic beta-cell may be observed by detection of circulating islet cell autoantibodies [“type 1A diabetes mellitus”], i.e., at least one of: GAD65 [glutamic acid decarboxylase-65], ICA [islet-cell cytoplasm], IA-2 [intracytoplasmatic domain of the tyrosine phosphatase-like protein IA-2], ZnT8 [zinc-transporter-8] or anti-insulin; or other signs of autoimmunity without the presence of typical circulating autoantibodies [type 1B diabetes], i.e. as detected through pancreatic biopsy or imaging). Typically a genetic predisposition is present (e.g. HLA, INS VNTR and PTPN22), but this is not always the case.

The term “type 2 diabetes mellitus” or “T2DM” is defined as the condition in which a subject has a fasting blood glucose or serum glucose concentration greater than 125 mg/dl (6.94 mmol/L). The measurement of blood glucose values is a standard procedure in routine medical analysis. If a glucose tolerance test is carried out, the blood sugar level of a diabetic will be in excess of 200 mg of glucose per dL (11.1 mmol/l) of plasma 2 hours after 75 g of glucose have been taken on an empty stomach. In a glucose tolerance test 75 g of glucose are administered orally to the patient being tested after 10-12 hours of fasting and the blood sugar level is recorded immediately before taking the glucose and 1 and 2 hours after taking it. In a healthy subject, the blood sugar level before taking the glucose will be between 60 and 110 mg per dL of plasma, less than 200 mg per dL 1 hour after taking the glucose and less than 140 mg per dL after 2 hours. If after 2 hours the value is between 140 and 200 mg, this is regarded as abnormal glucose tolerance.

The term “late stage type 2 diabetes mellitus” includes patients with a secondary drug failure, indication for insulin therapy and progression to micro- and macrovascular complications e.g. diabetic nephropathy, or coronary heart disease (CHD).

The term “HbA1c” refers to the product of a non-enzymatic glycation of the haemoglobin B chain. Its determination is well known to one skilled in the art. In monitoring the treatment of diabetes mellitus the HbA1c value is of exceptional importance. As its production depends essentially on the blood sugar level and the life of the erythrocytes, the HbA1c in the sense of a “blood sugar memory” reflects the average blood sugar levels of the preceding 4-6 weeks. Diabetic patients whose HbA1c value is consistently well adjusted by intensive diabetes treatment (i.e. <6.5% of the total haemoglobin in the sample), are significantly better protected against diabetic microangiopathy. For example, metformin on its own achieves an average improvement in the HbA1c value in the diabetic of the order of 1.0-1.5%. This reduction of the HbA1C value is not sufficient in all diabetics to achieve the desired target range of <7% or <6.5% and preferably <6% HbA1c.

The term “insufficient glycemic control” or “inadequate glycemic control” in the scope of the present invention means a condition wherein patients show HbA1c values above 6.5%, in particular above 7.0%, even more preferably above 7.5%, especially above 8%.

The “metabolic syndrome”, also called “syndrome X” (when used in the context of a metabolic disorder), also called the “dysmetabolic syndrome” is a syndrome complex with the cardinal feature being insulin resistance (Laaksonen D E, et al. Am J Epidemiol 2002; 156: 1070-7). According to the ATP III/NCEP guidelines (Executive Summary of the Third Report of the National Cholesterol Education Program (NCEP) Expert Panel on Detection, Evaluation, and Treatment of High Blood Cholesterol in Adults (Adult Treatment Panel III) JAMA: Journal of the American Medical Association (2001) 285: 2486-2497), diagnosis of the metabolic syndrome is made when three or more of the following risk factors are present:
1. Abdominal obesity, defined as waist circumference >40 inches or 102 cm in men, and >35 inches or 94 cm in women; or with regard to a Japanese ethnicity or Japanese patients defined as waist circumference ≥85 cm in men and ≥90 cm in women;
2. Triglycerides: ≥150 mg/dl
3. HDL-cholesterol <40 mg/dl in men
4. Blood pressure ≥130/85 mm Hg (SBP ≥130 or DBP ≥85)
5. Fasting blood glucose ≥100 mg/dL

The NCEP definitions have been validated (Laaksonen DE, et al. Am J Epidemiol. (2002) 156: 1070-7). Triglycerides and HDL cholesterol in the blood can also be determined by standard methods in medical analysis and are described for example in Thomas L (Editor): “Labor und Diagnose”, TH-Books Verlagsgesellschaft mbH, Frankfurt/Main, 2000.

According to a commonly used definition, hypertension is diagnosed if the systolic blood pressure (SBP) exceeds a value of 140 mm Hg and diastolic blood pressure (DBP) exceeds a value of 90 mm Hg. If a patient is diagnosed with diabetes it is currently recommended that the systolic blood pressure be reduced to a level below 130 mm Hg and the diastolic blood pressure be lowered to below 80 mm Hg.

The term “chronic kidney disease (CDK)” is defined as abnormalities of kidney structure or function, present for more than three months, with implications for health. CKD is classified based on cause, GFR category, and albuminuria category (CGA).

CKD has been classified into 5 stages, where stage 1 is kidney damage with normal GFR (mL/min/1.73 m2) of 90 or above; stage 2 is kidney damage with a mild decrease in GFR (GFR 60-89); stage 3 is a moderate decrease in GFR (GFR 30-59); stage 4 is a severe decrease in GFR (GFR 15-29); and stage 5 is kidney failure (GFR <15 or dialysis). Stage 3 has been subdivided into stage 3A, which is a mild to moderate decrease in GFR (GFR 45-59), and stage 3B, which is a moderate to severe decrease in GFR (GFR 30-44).

The term “glomerular filtration rate (GFR)” is defined as the volume of fluid filtered from the renal (kidney) glomerular capillaries into the Bowman's capsule per unit time. It is indicative of overall kidney function. The glomerular filtration rate (GFR) can be calculated by measuring any chemical that has a steady level in the blood, and is freely filtered but neither reabsorbed nor secreted by the kidneys. The rate therefore measured is the quantity of the substance in the urine that originated from a calculable volume of blood. The GFR is typically recorded in units of volume per time, e.g., milliliters per minute and the formula below can be used:

GFR=(Urine Concentration×Urine Volume)/Plasma Concentration

The GFR can be determined by injecting inulin into the plasma. Since inulin is neither reabsorbed nor secreted by the kidney after glomerular filtration, its rate of excretion is directly proportional to the rate of filtration of water and solutes across the glomerular filter. A normal value is: GFR=90-125 mL/min/1.73 m², in particular GFR=100-125 mL/min/1.73 m².

Other principles to determine GFR involve measuring 51Cr-EDTA, [125l]iothalamate or iohexol.

The “estimated glomerular filtration rate (eGFR)” is defined as derived at screening from serum creatinine values based on e.g., the Chronic Kidney Disease Epidemiology Collaboration (CKD-EPI) equation, the Cockcroft-Gault formula or the Modification of Diet in Renal Disease (MDRD) formula, which are all known in the art.

The term “albuminuria” is defined as a condition wherein more than the normal amount of albumin is present in the urine. Albuminuria can be determined by the albumin excretion rate (AER) and/or the albumin-to-creatine ratio (ACR) in the urine (also referred to as UACR).

Albuminuria categories in CKD are defined as follows:

| | | ACR (approximate equivalent) | | |
|---|---|---|---|---|
| Category | AER (mg/24 hours) | (mg/mmol) | (mg/g) | Terms |
| A1 | <3 | <3 | <30 | Normal to mildly |
| | | | | increased |
| A2 | 30-300 | 3-30 | 30-300 | Moderately increased |
| A3 | >300 | >30 | >300 | Severely increased |

Category A1 reflects no albuminuria, category A2 reflects microalbuminuria, category A3 reflects macroalbuminuria. The progression of category A1 usually leads to microalbuminuria (A2) but may also directly result in macroalbuminuria (A3). Progression of microalbuminuria (A2) results in macroalbuminuria (A3).

The term “empagliflozin” refers to the SGLT2 inhibitor 1-chloro-4-(B-D-glucopyranos-1-yl)-2-[4-((S)-tetrahydrofuran-3-yloxy)-benzyl]-benzene of the formula

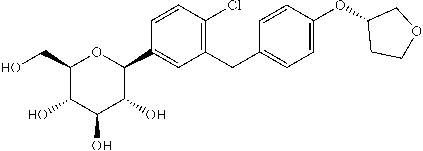 [SMILES: OC[C@H]1O[C@@H](c2ccc(Cl)c(Cc3ccc(O[C@H]4CCOC4)cc3)c2)[C@H](O)[C@@H](O)[C@@H]1O]

as described for example in WO 2005/092877. Methods of synthesis are described in the literature, for example WO 06/120208 and WO 2011/039108. According to this invention, it is to be understood that the definition of empagliflozin also comprises its hydrates, solvates and polymorphic forms thereof, and prodrugs thereof. An advantageous crystalline form of empagliflozin is described in WO 2006/117359 and WO 2011/039107 which hereby are incorporated herein in their entirety. This crystalline form possesses good solubility properties which enables a good bioavailability of the SGLT2 inhibitor. Furthermore, the crystalline form is physico-chemically stable and thus provides a good shelf-life stability of the pharmaceutical composition. Preferred pharmaceutical compositions, such as solid formulations for oral administration, for example tablets, are described in WO 2010/092126, which hereby is incorporated herein in its entirety.

The terms “treatment” and “treating” comprise therapeutic treatment of patients having already developed said condition, in particular in manifest form. Therapeutic treatment may be symptomatic treatment in order to relieve the symptoms of the specific indication or causal treatment in order to reverse or partially reverse the conditions of the indication or to stop or slow down progression of the disease. Thus the compositions and methods of the present invention may be used for instance as therapeutic treatment over a period of time as well as for chronic therapy.

The terms “prophylactically treating”, “preventivally treating” and “preventing” are used interchangeably and comprise a treatment of patients at risk to develop a condition mentioned hereinbefore, thus reducing said risk.

The term “tablet” comprises tablets without a coating and tablets with one or more coatings. Furthermore the “term” tablet comprises tablets having one, two, three or even more layers and press-coated tablets, wherein each of the beforementioned types of tablets may be without or with one or more coatings. The term “tablet” also comprises mini, melt, chewable, effervescent and orally disintegrating tablets.

The terms “pharmacopoe” and “pharmacopoeias” refer to standard pharmacopoeias such as the “USP 31-NF 26 through Second Supplement” (United States Pharmacopeial Convention) or the “European Pharmacopoeia 6.3” (European Directorate for the Quality of Medicines and Health Care, 2000-2009).

### DETAILED DESCRIPTION OF THE INVENTION

In one aspect, the present invention relates to certain SGLT-2 inhibitors, for example empagliflozin, for use in treating, preventing, protecting against, reducing the risk of, delaying the occurrence of and/or delaying the progression of chronic kidney disease in patients, for example patients with prediabetes, type 1 or type 2 diabetes mellitus.

In a further aspect, the present invention relates to certain SGLT-2 inhibitors, for example empagliflozin, for use in treating, preventing, protecting against or delaying new onset of albuminuria in patients.

In a further aspect, the present invention relates to certain SGLT-2 inhibitors, for example empagliflozin, for use in treating, preventing, protecting against or delaying the progression from no albuminuria to micro- or macroalbuminuria in a patient at risk for renal disease.

In a further aspect, the present invention relates to certain SGLT-2 inhibitors, for example empagliflozin, for use in treating, preventing, protecting against or delaying the progression from microalbuminuria to macroalbuminuria in a patient with chronic kidney disease.

In a further aspect, the present invention relates to certain SGLT-2 inhibitors, for example empagliflozin, for use in treating, preventing, protecting against or delaying the progression of chronic kidney disease in a patient with chronic kidney disease.

In a further aspect, the present invention relates to certain SGLT-2 inhibitors, for example empagliflozin, for use in treating, preventing, protecting against or delaying the occurrence of:
new onset of albuminuria,
doubling of serum creatinine level accompanied by an eGFR (based on modification of diet in renal disease (MDRD) formula) ≤45 mL/min/1.73 m²,
need for continuous renal replacement therapy, or
death due to renal disease.

In one aspect, a patient according of the present invention is a patient with prediabetes, type 1 or type 2 diabetes mellitus. In one aspect, a patient according to the present invention is a patient is a patient at risk for renal disease. In one aspect, a patient according to the present invention is a patient with or at risk of a cardiovascular disease. In one aspect, a patient according to the present invention is a patient with prediabetes, type 1 or type 2 diabetes mellitus and with or at risk of a cardiovascular disease.

In one aspect of the present invention, in a method or use disclosed herein a patient is a patient with obesity-related Glomerulopathy, a patient with perihilar fokal-segmental glomerulosclerosis or a patient with IgA nephropathy.

Accordingly, in one embodiment, the present invention provides a method of treating, preventing, protecting against, reducing the risk of, delaying the occurrence of and/or delaying the progression of chronic kidney disease in a patient with obesity-related

Glomerulopathy, in a patient with perihilar fokal-segmental glomerulosclerosis or in a patient with IgA nephropathy.

SGLT2 inhibitors (sodium-glucose co-transporter 2) represent a novel class of agents that are being developed for the treatment or improvement in glycemic control in patients with type 2 diabetes. Glucopyranosyl-substituted benzene derivative are described as SGLT2 inhibitors, for example in WO 01/27128, WO 03/099836, WO 2005/092877, WO 2006/034489, WO 2006/064033, WO 2006/117359, WO 2006/117360, WO 2007/025943, WO 2007/028814, WO 2007/031548, WO 2007/093610, WO 2007/128749, WO 2008/049923, WO 2008/055870, WO 2008/055940. The glucopyranosyl-substituted benzene derivatives are proposed as inducers of urinary sugar excretion and as medicaments in the treatment of diabetes.

Renal filtration and reuptake of glucose contributes, among other mechanisms, to the steady state plasma glucose concentration and can therefore serve as an antidiabetic target.

Reuptake of filtered glucose across epithelial cells of the kidney proceeds via sodium-dependent glucose cotransporters (SGLTs) located in the brush-border membranes in the tubuli along the sodium gradient. There are at least 3 SGLT isoforms that differ in their expression pattern as well as in their physico-chemical properties. SGLT2 is exclusively expressed in the kidney, whereas SGLT1 is expressed additionally in other tissues like intestine, colon, skeletal and cardiac muscle. SGLT3 has been found to be a glucose sensor in interstitial cells of the intestine without any transport function. Potentially, other related, but not yet characterized genes, may contribute further to renal glucose reuptake. Under normoglycemia, glucose is completely reabsorbed by SGLTs in the kidney, whereas the reuptake capacity of the kidney is saturated at glucose concentrations higher than 10 mM, resulting in glucosuria (“diabetes mellitus”). This threshold concentration can be decreased by SGLT2-inhibition. It has been shown in experiments with the SGLT inhibitor phlorizin that SGLT-inhibition will partially inhibit the reuptake of glucose from the glomerular filtrate into the blood leading to a decrease in blood glucose concentration and to glucosuria.

Empagliflozin is a novel SGLT2 inhibitor that is described for the treatment or improvement in glycemic control in patients with type 2 diabetes mellitus, for example in WO 05/092877, WO 06/117359, WO 06/120208, WO 2010/092126, WO 2010/092123, WO 2011/039107, WO 2011/039108.

Accordingly, in a particular embodiment, a SGLT-2 inhibitor within the meaning of this invention is empagliflozin.

Further, the present invention relates to therapeutic (treatment or prevention) methods as described herein, in particular methods for the prevention or treatment of renal diseases, said method comprising administering an effective amount of a SGLT-2 inhibitor as described herein and, optionally, one or more other active or therapeutic agents as described herein to the patient in need thereof.

Chronic kidney disease (CKD), also known as chronic renal disease, is a progressive loss in renal function over a period of months or years. Patients with renal disease, renal dysfunction or renal impairment may include patients with chronic renal insufficiency or impairment, which can be stratified (if not otherwise noted) according to glomerular filtration rate (GFR, ml/min/1.73 m²) into 5 disease stages: stage 1 characterized by normal GFR≥90 plus either persistent albuminuria (e.g. UACR≥30 mg/g) or known structural or hereditary renal disease; stage 2 characterized by mild reduction of GFR (GFR 60-89) describing mild renal impairment; stage 3 characterized by moderate reduction of GFR (GFR 30-59) describing moderate renal impairment; stage 4 characterized by severe reduction of GFR (GFR 15-29) describing severe renal impairment; and terminal stage 5 characterized by requiring dialysis or GFR<15 describing established kidney failure (end-stage renal disease, ESRD).

Chronic kidney disease and its stages (CKD 1-5) can be usually characterized or classified accordingly, such as based on the presence of either kidney damage (albuminuria) or impaired estimated glomerular filtration rate (GFR<60 [ml/min/1.73 m²], with or without kidney damage).

For the purpose of the present invention, the estimated glomerular filtration rate (eGFR) is derived from the serum creatinine (SCr) value based on the MDRD formula below:

eGFR (mL/min/1.73 m²)=175×[SCr (μmol/L)/88.4]−1.154×[age]−0.203×[0.742 if patient is female]×[1.212 if patient is of African origin]

For additional analyses, renal function can also be classified by the estimated creatinine clearance rate (eCCr) value, based on the Cockcroft-Gault formula below:

eCCr (mL/min)=(140−age)×(weight in kg)×[0.85 if patient is female]/(72×SCr (mg/dL))

Renal function classification based on eCCr is similar to the eGFR classification: normal renal function (≥90 mL/min), mild impairment (60 to <90 mL/min), moderate impairment (30 to <60 mL/min), and severe impairment (≥15 to <30 mL/min).

Generally, mild renal impairment according to the present invention corresponds to stage 2 chronic kidney disease, moderate renal impairment according to the present invention generally corresponds to stage 3 chronic kidney disease, and severe renal impairment according to the present invention generally corresponds to stage 4 chronic kidney disease. Likewise, moderate A renal impairment according to the present invention generally corresponds to stage 3A chronic kidney disease and moderate B renal impairment according to the present invention generally corresponds to stage 3B chronic kidney disease.

Accordingly, in one aspect, the present invention relates to certain SGLT-2 inhibitors, for example empagliflozin, for use in treating, preventing, protecting against or delaying the progression of chronic kidney disease in a patient, in particular a patient according to the present invention, for example the progression from stage 1 chronic kidney disease to stage 2 chronic kidney disease, for example the progression from stage 2 chronic kidney disease to stage 3 chronic kidney disease, for example the progression from stage 3 chronic kidney disease to stage 4 chronic kidney disease, for example the progression from stage 4 chronic kidney disease to stage 5 chronic kidney disease.

In a further aspect of the present invention, the progression of chronic kidney disease in a patient is for example the progression from stage 2 chronic kidney disease to stage 3A chronic kidney disease, for example the progression from stage 3A chronic kidney disease to stage 3B chronic kidney disease, for example the progression from stage 3B chronic kidney disease to stage 4 chronic kidney disease.

In a further aspect of the present invention, the progression of chronic kidney disease in a patient is for example the progression from stage 2 chronic kidney disease to stage 4 or 5 chronic kidney disease, for example the progression from stage 3 chronic kidney disease to stage 5 chronic kidney disease, for example the progression from stage 3A or 3B chronic kidney disease to stage 5 chronic kidney disease.

In a further aspect, a patient with chronic kidney disease according to the present invention is a patient with stage 1 chronic kidney disease, stage 2 chronic kidney disease, stage 3 chronic kidney disease, stage 4 chronic kidney disease, or stage 5 chronic kidney disease. In a further aspect, a patient with chronic kidney disease according to the present invention is a patient with stage 3A chronic kidney disease or stage 3B chronic kidney disease,

In some aspects, renal disease, renal dysfunction, or insufficiency or impairment of renal function (including mild, moderate and/or severe renal impairment) may also be suggested (if not otherwise noted) by elevated serum creatinine levels (e.g. serum creatinine levels above the upper limit of normal for their age, e.g. ≥130-150 μmol/l, or ≥1.5 mg/dl (≥136 μmol/l) in men and ≥1.4 mg/dl (≥124 μmol/l) in women) or abnormal creatinine clearance (e.g. glomerular filtration rate (GFR)≤30-60 ml/min).

In some further aspects, mild renal impairment may be also suggested (if not otherwise noted) by a creatinine clearance of 50-80 ml/min (approximately corresponding to serum creatine levels of ≤1.7 mg/dl in men and ≤1.5 mg/dl in women); moderate renal impairment may be e.g. suggested (if not otherwise noted) by a creatinine clearance of 30-50 ml/min (approximately corresponding to serum creatinine levels of >1.7 to ≤3.0 mg/dl in men and >1.5 to ≤2.5 mg/dL in women); and severe renal impairment may be e.g. suggested (if not otherwise noted) by a creatinine clearance of <30 ml/min (approximately corresponding to serum creatinine levels of >3.0 mg/dl in men and >2.5 mg/dl in women). Patients with end-stage renal disease require dialysis (e.g. hemodialysis or peritoneal dialysis).

In some further aspects, albuminuria can also be a sign of kidney disease. Albuminuria stages may be classified as disclosed herein, and patients may be stratified in category A1, which reflects no albuminuria, category A2, which reflects microalbuminuria, and category A3, which reflects macroalbuminuria.

Accordingly, in a further aspect, a patient with chronic kidney disease according to the present invention is a patient with microalbuminuria or with macroalbuminuria.

In one aspect of the present invention, it has been found that empagliflozin has nephroprotective properties, in particular as described herein. In particular, it has been shown that administration of empagliflozin has the property to maintain or improve renal function over time in certain patient group, for example as described herein, as demonstrated after discontinuation of administration of empagliflozin.

In one aspect, a patient in the context of the present invention is a patient at risk of renal disease. A patient at risk of renal disease is for example a patient with at least one of the following:
prediabetes, type 1 or 2 diabetes mellitus,
hypertension,
metabolic syndrome,
cardiovascular disease.

In one aspect, a patient in the context of the present invention is a patient with prediabetes, type 1 or 2 diabetes mellitus.

Type 2 diabetes mellitus is a common chronic and progressive disease arising from a complex pathophysiology involving the dual endocrine effects of insulin resistance and impaired insulin secretion with the consequence not meeting the required demands to maintain plasma glucose levels in the normal range. This leads to hyperglycemia and its associated micro- and macrovascular complications or chronic damages, such as e.g. diabetic nephropathy, retinopathy or neuropathy, or macrovascular (e.g. cardiovascular) complications. The vascular disease component plays a significant role, but is not the only factor in the spectrum of diabetes associated disorders. The high frequency of complications leads to a significant reduction of life expectancy. Diabetes is currently the most frequent cause of adult-onset loss of vision, renal failure, and amputation in the Industrialised World because of diabetes induced complications and is associated with a two to five fold increase in cardiovascular disease risk. Type 1 diabetes mellitus (Type 1 diabetes), also called insulin dependent diabetes mellitus or juvenile diabetes, is a form of diabetes mellitus that results from autoimmune destruction of insulin-producing beta cells of the pancreas. The subsequent lack of insulin leads to increased blood glucose concentrations and increased urinary glucose excretion. The classical symptoms are polyuria, polydipsia, polyphagia, and weight loss. Type 1 diabetes may be fatal unless treated with insulin. Complications from type 1 diabetes are the same or similar to complications from type 2 diabetes.

Large randomized studies have established that intensive and tight glycemic control during early (newly diagnoses to 5 years) stage diabetes has enduring beneficial effects and reduces the risk of diabetic complications, both micro- and macrovascular. However, many patients with diabetes still develop diabetic complications despite receiving intensified glycemic control.

Standard therapy of type 1 diabetes is insulin treatment. Therapies for type 1 diabetes are for example described in WO 2012/062698.

The treatment of type 2 diabetes typically begins with diet and exercise, followed by oral antidiabetic monotherapy, and although conventional monotherapy may initially control blood glucose in some patients, it is however associated with a high secondary failure rate. The limitations of single-agent therapy for maintaining glycemic control may be overcome, at least in some patients, and for a limited period of time by combining multiple drugs to achieve reductions in blood glucose that cannot be sustained during long-term therapy with single agents. Available data support the conclusion that in most patients with type 2 diabetes current monotherapy will fail and treatment with multiple drugs will be required. But, because type 2 diabetes is a progressive disease, even patients with good initial responses to conventional combination therapy will eventually require an increase of the dosage or further treatment with insulin because the blood glucose level is very difficult to maintain stable for a long period of time. Although existing combination therapy has the potential to enhance glycemic control, it is not without limitations (especially with regard to long term efficacy). Further, traditional therapies may show an increased risk for side effects, such as hypoglycemia or weight gain, which may compromise their efficacy and acceptability.

Oral antidiabetic drugs conventionally used in therapy (such as e.g. first- or second-line, and/or mono- or (initial or add-on) combination therapy) include, without being restricted thereto, metformin, sulphonylureas, thiazolidinediones, DPP-4 inhibitors, glinides and α-glucosidase inhibitors.

Non-oral (typically injected) antidiabetic drugs conventionally used in therapy (such as e.g. first- or second-line, and/or mono- or (initial or add-on) combination therapy) include, without being restricted thereto, GLP-1 or GLP-1 analogues, and insulin or insulin analogues.

The SGLT2 inhibitor therein also exhibits a very good efficacy with regard to glycemic control, in particular in view of a reduction of fasting plasma glucose, postprandial plasma glucose and/or glycosylated hemoglobin (HbA1c). By administering a pharmaceutical composition according to this invention, a reduction of HbA1c equal to or greater than preferably 0.5%, even more preferably equal to or greater than 1.0% can be achieved and the reduction is particularly in the range from 1.0% to 2.0%.

In a further embodiment, a patient according to the present invention is a patient who shows one, two or more of the following conditions:

(a) a fasting blood glucose or serum glucose concentration greater than 100 mg/dl, in particular greater than 125 mg/dL;

(b) a postprandial plasma glucose equal to or greater than 140 mg/dL;

(c) an HbA1c value equal to or greater than 6.5%, in particular equal to or greater than 7.0%, especially equal to or greater than 7.5%, even more particularly equal to or greater than 8.0%.

In a further embodiment, a patient according to the present invention is a patient who shows one, two or more of the following conditions:

(a) insufficient glycemic control with diet and exercise alone;

(b) insufficient glycemic control despite oral monotherapy with metformin, in particular despite oral monotherapy at a maximal tolerated dose of metformin;

(c) insufficient glycemic control despite oral monotherapy with one or more other antidiabetic agent, in particular despite oral monotherapy at a maximal tolerated dose of the other antidiabetic agent.

In a further embodiment, a patient according to the present invention is a patient who shows one, two or more of the following conditions:

(a) obesity (including class I, II and/or III obesity), visceral obesity and/or abdominal obesity,

(b) triglyceride blood level ≥150 mg/dL,

(c) HDL-cholesterol blood level <40 mg/dL in female patients and <50 mg/dl in male patients,

(d) a systolic blood pressure ≥130 mm Hg and a diastolic blood pressure ≥85 mm Hg,

(e) a systolic blood pressure ≥130 mm Hg and a diastolic blood pressure ≥80 mm Hg,

(f) a fasting blood glucose level ≥100 mg/dL.

In one embodiment, a patient according to the present invention is a patient with prediabetes, type 1 or 2 diabetes mellitus and hypertension. In one embodiment, a patient according to the present invention is a patient with prediabetes, type 1 or 2 diabetes mellitus and a systolic blood pressure ≥130 mm Hg and a diastolic blood pressure ≥80 mm Hg.

In one embodiment, diabetes patients within the meaning of this invention may include patients who have not previously been treated with an antidiabetic drug (drug-naïve patients). Thus, in an embodiment, the therapies described herein may be used in naïve patients. In another embodiment, diabetes patients within the meaning of this invention may include patients with advanced or late stage type 2 diabetes mellitus (including patients with failure to conventional antidiabetic therapy), such as e.g. patients with inadequate glycemic control on one, two or more conventional oral and/or non-oral antidiabetic drugs as defined herein, such as e.g. patients with insufficient glycemic control despite (mono-)therapy with metformin, a thiazolidinedione (particularly pioglitazone), a sulphonylurea, a glinide, a DPP-4 inhibitor, GLP-1 or GLP-1 analogue, insulin or insulin analogue, or an α-glucosidase inhibitor, or despite dual combination therapy with metformin/sulphonylurea, metformin/thiazolidinedione (particularly pioglitazone), metformin/DPP-4 inhibitor, sulphonylurea/α-glucosidase inhibitor, pioglitazone/sulphonylurea, metformin/insulin, pioglitazone/insulin or sulphonylurea/insulin.

In one embodiment, a patient according to the present invention is a patient receiving treatment with a non-oral antidiabetic drug, for example GLP1-analog (for example short acting GLP-1 analog such as exenatide, liraglutide or lixisenatide, or long-acting GLP-1 analog such as exenatide extended-release, albiglutide or dulaglutide), for example insulin or insulin analogue, for example basal insulin, such as glargine, detemir and/or NPH insulin.

In one embodiment, a patient according to the present invention is a patient receiving treatment with insulin or insulin analogue. An insulin or insulin analogue may include normal insulin, human insulin, insulin derivatives, zinc insulins and insulin analogues, including formulations thereof with modified release profiles, in particular as used in the therapy of humans. The insulin may be selected from the group consisting of:
rapid-acting insulins,
short-acting insulins,
intermediate-acting insulins,
long-acting insulins,

and mixtures thereof.

Mixtures of insulins may comprise mixtures of short- or rapid-acting insulins with long-acting insulins. For example such mixtures are marketed as Actraphane/Mixtard or Novomix.

The term “insulin” in the scope of the present invention covers insulins as described hereinbefore and hereinafter which are administered to the patient via injection, preferably subcutaneous injection, via infusion, including pumps, via inhalation or other routes of administration. Insulins to be administered via inhalation are for example Exubera (Pfizer), AIR (Lilly) and AER (Novo Nordisk).

Rapid-acting insulins usually start lowering the blood glucose within about 5 to 15 minutes and are effective for about 3 to 4 hours. Examples of rapid-acting insulins are insulin aspart, insulin lispro and insulin glulisine. Insulin Lispro is marketed under the trade name Humalog and Liprolog. Insulin Aspart is marketed under the trade names NovoLog and NovoRapid. Insulin glulisine is marketed under the trade name Apidra.

Short-acting insulins usually start lowering the blood glucose within about 30 minutes and are effective about 5 to 8 hours. An example is regular insulin or human insulin.

Intermediate-acting insulins usually start lowering the blood glucose within about 1 to 3 hours and are effective for about 16 to 24 hours. An example is NPH insulin, also known as Humulin N, Novolin N, Novolin NPH and isophane insulin. Another example are lente insulins, such as Semilente or Monotard.

Long-acting insulins usually start lowering the blood glucose within 1 to 6 hours and are effective for up to about 24 hours or even up to or beyond 32 hours. Long-acting insulin usually provides a continuous level of insulin activity (for up to 24-36 hours) and usually operates at a maximum strength (with flat action profile) after about 8-12 hours, sometimes longer. Long-acting insulin is usually administered in the morning or before bed. Examples of long-acting insulin may include, but are not limited to, insulin glargine, insulin detemir or insulin degludec, which are insulin analogues, and ultralente insulin, which is regular human insulin formulated for slow absorption. Long-acting insulin is suited to provide for basal, as opposed to prandial, insulin requirements (e.g. to control hyperglycemia). Long-acting insulin may be typically administered ranging from twice or once daily, over thrice weekly up to once weekly (ultra long-acting insulin). Insulin glargine is marketed under the trade name Lantus for example. Insulin detemir is marketed under the tradename Levemir for example.

In one embodiment, a long-acting insulin is an acylated derivative of human insulin. Acylated insulin derivatives may be such wherein a lipophilic group is attached to the lysine residue in position B29. A commercial product is Levemir® comprising Lys^{B29}(N^{ε}-tetradecanoyl) des(B30) human insulin (insulin detemir). Another example is N^{εB29}-(N^{α}-(ω-carboxypentadecanoyl)-L-γ-glutamyl) des(B30) human insulin (insulin degludec).

In one embodiment, a long-acting insulin is such comprising positively charged amino acids such as Arg attached to the C-terminal end of the B-chain. A commercial product is Lantus® (insulin glargine) comprising Gly^{A21}, Arg^{B31}, Arg^{B32} human insulin.

In one embodiment, a patient according to the present invention is a patient receiving treatment with a mixture of insulin and GLP-1 analog, for example a mixture of insulin glargine and lixisenatide.

In a further aspect, a patient according to the present invention is a patient with or at risk of a cardiovascular disease.

In one embodiment, the patient is a patient with one or more cardiovascular risk factors selected from A), B), C) and D), for example a patient with type 1 or type 2 diabetes mellitus or with pre-diabetes with one or more cardiovascular risk factors selected from A), B), C) and D):

A) previous or existing vascular disease selected from myocardial infarction, coronary artery disease, percutaneous coronary intervention, coronary artery by-pass grafting, ischemic or hemorrhagic stroke, congestive heart failure, and peripheral occlusive arterial disease,

B) advanced age >/=60-70 years, and

C) one or more cardiovascular risk factors selected from
advanced type 2 diabetes mellitus >10 years duration,
hypertension,
current daily cigarette smoking,
dyslipidemia,
obesity,
age >/=40,
metabolic syndrome, hyperinsulinemia or insulin resistance, and
hyperuricemia, erectile dysfunction, polycystic ovary syndrome, sleep apnea, or family history of vascular disease or cardiomyopathy in first-degree relative;

D) one or more of the following:
confirmed history of myocardial infarction,
unstable angina with documented multivessel coronary disease or positive stress test,
multivessel Percutaneous Coronary Intervention,
multivessel Coronary Artery By-pass Grafting (CABG),
history of ischemic or hemorrhagic stroke,
peripheral occlusive arterial disease.

In a further aspect of the present invention, a patient having cardiovascular disease is defined as having at least one of the following:
Confirmed history of myocardial infarction; or
Evidence of multivessel coronary artery disease, in 2 or more major coronary arteries, irrespective of the revascularization status, i.e.
   a) Either the presence of a significant stenosis (imaging evidence of at least 50% narrowing of the luminal diameter measured during a coronary angiography or a multi-sliced computed tomography angiography), in 2 or more major coronary arteries,
   b) Or a previous revascularisation (percutaneous transluminal coronary angioplasty with or without stent, or coronary artery bypass grafting), in 2 or more major coronary arteries,
   c) Or the combination of previous revascularisation in one major coronary artery (percutaneous transluminal coronary angioplasty with or without stent, or coronary artery bypass grafting), and the presence of a significant stenosis in another major coronary artery (imaging evidence of at least 50% narrowing of the luminal diameter measured during a coronary angiography or a multi-sliced computed tomography angiography),
      Note: A disease affecting the left main coronary artery is considered as a 2-vessel disease.
Evidence of a single vessel coronary artery disease with:
   a) The presence of a significant stenosis i.e. the imaging evidence of at least 50% narrowing of the luminal diameter of one major coronary artery in patients not subsequently successfully revascularised (measured during a coronary angiography or a multi-sliced computed tomography angiography)
   b) And at least one of the following (either (i) or (ii)):
      i. A positive non invasive stress test, confirmed by either:
         1. A positive exercise tolerance test in patients without a complete left bundle branch block, Wolff-Parkinson-White syndrome, or paced ventricular rhythm, or
         2. A positive stress echocardiography showing regional systolic wall motion abnormalities, or
         3. A positive scintigraphic test showing stress-induced ischemia, i.e. the development of transient perfusion defects during myocardial perfusion imaging;
      ii. Or patient discharged from hospital with a documented diagnosis of unstable angina within 12 months prior to selection.
Episode of unstable angina with confirmed evidence of coronary multivessel or single vessel disease as defined above.
History of ischemic or haemorrhagic stroke
Presence of peripheral artery disease (symptomatic or not) documented by either: previous limb angioplasty, stenting or bypass surgery; or previous limb or foot amputation due to circulatory insufficiency; or angiographic evidence of significant (>50%) peripheral artery stenosis in at least one limb; or evidence from a non-invasive measurement of significant (>50% or as reported as hemodynamically significant) peripheral artery stenosis in at least one limb; or ankle brachial index of <0.9 in at least one limb.

In a further aspect of the present invention, a patient having cardiovascular disease is defined as having at least one of the following:
a) Confirmed history of myocardial infarction,
b) Unstable angina with documented multivessel coronary disease (at least two major coronary arteries in angiogram) or positive stress test (ST segment depression >=2 mm or a positive nuclear perfusion scintigram),
c) Multivessel Percutaneous Coronary Intervention (PCI),
d) Multivessel Coronary Artery By-pass Grafting (CABG), including with recurrent angina following surgery,
e) History of ischemic or hemorrhagic stroke,
f) Peripheral occlusive arterial disease (previous limb bypass surgery or percutaneous transluminal angioplasty; previous limb or foot amputation due to circulatory insufficiency, angiographic or imaging detected (for example: ultrasound, Magnetic Resonance Imaging) significant vessel stenosis of major limb arteries).

Accordingly, in one aspect, the present invention relates to a certain SGLT-2 inhibitor, in particular empagliflozin, for use in a method as described herein, in a patient with one or more risk factors selected from A), B), C) and D):

A) previous or existing vascular disease (such as e.g. myocardial infarction (e.g. silent or non-silent), coronary artery disease, percutaneous coronary intervention, coronary artery by-pass grafting, ischemic or hemorrhagic stroke, congestive heart failure (e.g. NYHA class I, II, III or IV, e.g. left ventricular function <40%), or peripheral occlusive arterial disease), B) advanced age (such as e.g. age >/=60-70 years), and C) one or more cardiovascular risk factors selected from
advanced type 1 or type 2 diabetes mellitus (such as e.g. >10 years duration),
hypertension (such as e.g. >130/80 mm Hg, or systolic blood pressure >140 mmHg or on at least one blood pressure lowering treatment),
current daily cigarette smoking,
dyslipidemia (such as e.g. atherogenic dyslipidemia, postprandial lipemia, or high level of LDL cholersterol (e.g. LDL cholesterol >/=130-135 mg/dL), low level of HDL cholesterol (e.g. <35-40 mg/dL in men or <45-50 mg/dL in women) and/or high level of triglycerides (e.g. >200-400 mg/dL) in the blood, or on at least one treatment for lipid abnormality),
obesity (such as e.g. abdominal and/or visceral obesity, or body mass index >/=45 kg/m2),
age >/=40,
metabolic syndrome, hyperinsulinemia or insulin resistance, and
hyperuricemia, erectile dysfunction, polycystic ovary syndrome, sleep apnea, or family history of vascular disease or cardiomyopathy in first-degree relative,

D) one or more of the following:
confirmed history of myocardial infarction,
unstable angina with documented multivessel coronary disease or positive stress test,
multivessel Percutaneous Coronary Intervention,
multivessel Coronary Artery By-pass Grafting (CABG),
history of ischemic or hemorrhagic stroke,
peripheral occlusive arterial disease.

said method comprising administering a therapeutically effective amount of the SGLT-2 inhibitor, optionally in combination with one or more other therapeutic substances, to the patient.

In one aspect, a patient in the context of the present invention is a patient with hypertension. In one aspect, a patient in the context of the present invention is a patient with metabolic syndrome.

The present invention further relates to a pharmaceutical composition comprising a certain SGLT-2 inhibitor as defined herein, empagliflozin, for use in the therapies described herein, for example in a patient or patient group as described herein.

When this invention refers to patients requiring treatment or prevention, it relates primarily to treatment and prevention in humans, but the pharmaceutical composition may also be used accordingly in veterinary medicine in mammals. In the scope of this invention adult patients are preferably humans of the age of 18 years or older. Also in the scope of this invention, patients are adolescent humans, i.e. humans of age 10 to 17 years, preferably of age 13 to 17 years.

In a further aspect, a method according to the present invention further comprises improving glycemic control in patients having type 1 or type 2 diabetes mellitus or showing first signs of pre-diabetes.

In a further aspect, a method according to the present invention further comprises improving glycemic control and/or for reducing of fasting plasma glucose, of postprandial plasma glucose and/or of glycosylated hemoglobin HbA1c in a patient in need thereof who is diagnosed with impaired glucose tolerance (IGT), impaired fasting blood glucose (IFG) with insulin resistance, with metabolic syndrome and/or with type 2 or type 1 diabetes mellitus.

In a further aspect, a method according to the present invention further comprises improving glycemic control in patients, in particular in adult patients, with type 2 diabetes mellitus as an adjunct to diet and exercise.

Within the scope of the present invention it has now been found that certain SGLT-2 inhibitors as defined herein, optionally in combination with one or more other therapeutic substances (e.g. selected from those described herein), as well as pharmaceutical combinations, compositions or combined uses according to this invention of such SGLT-2 inhibitors as defined herein have properties, which make them suitable for the purpose of this invention and/or for fulfilling one or more of above needs. The present invention thus relates to a certain SGLT-2 inhibitor as defined herein, preferably empagliflozin, for use in the therapies described herein.

Furthermore, it can be found that the administration of a pharmaceutical composition according to this invention results in no risk or in a low risk of hypoglycemia. Therefore, a treatment or prophylaxis according to this invention is also advantageously possible in those patients showing or having an increased risk for hypoglycemia.

It will be appreciated that the amount of the pharmaceutical composition according to this invention to be administered to the patient and required for use in treatment or prophylaxis according to the present invention will vary with the route of administration, the nature and severity of the condition for which treatment or prophylaxis is required, the age, weight and condition of the patient, concomitant medication and will be ultimately at the discretion of the attendant physician. In general, however, the SGLT2 inhibitor according to this invention is included in the pharmaceutical composition or dosage form in an amount sufficient that by its administration the glycemic control in the patient to be treated is improved.

In the following preferred ranges of the amount of the SGLT2 inhibitor to be employed in the pharmaceutical composition and the methods and uses according to this invention are described. These ranges refer to the amounts to be administered per day with respect to an adult patient, in particular to a human being, for example of approximately 70 kg body weight, and can be adapted accordingly with regard to an administration 2, 3, 4 or more times daily and with regard to other routes of administration and with regard to the age of the patient. Within the scope of the present invention, the pharmaceutical composition is preferably administered orally. Other forms of administration are possible and described hereinafter. Preferably the one or more dosage forms comprising the SGLT2 inhibitor is oral or usually well known.

In general, the amount of the SGLT2 inhibitor in the pharmaceutical composition and methods according to this invention is preferably the amount usually recommended for a monotherapy using said SGLT2 inhibitor.

The preferred dosage range of the SGLT2 inhibitor is in the range from 0.5 mg to 200 mg, even more preferably from 1 to 100 mg, most preferably from 1 to 50 mg per day. In one aspect, a preferred dosage of the SGLT2 inhibitor empagliflozin is 10 mg or 25 mg per day. The oral administration is preferred. Therefore, a pharmaceutical composition may comprise the hereinbefore mentioned amounts, in particular from 1 to 50 mg or 1 to 25 mg. Particular dosage strengths (e.g. per tablet or capsule) are for example 1, 2.5, 5, 7.5, 10, 12.5, 15, 20, 25 or 50 mg of the SGLT2 inhibitor, in particular empagliflozin. In one aspect, a pharmaceutical composition comprises 10 mg or 25 mg of empagliflozin. The application of the active ingredient may occur up to three times a day, preferably one or two times a day, most preferably once a day.

A pharmaceutical composition which is present as a separate or multiple dosage form, preferably as a kit of parts, is useful in combination therapy to flexibly suit the individual therapeutic needs of the patient.

According to a first embodiment a preferred kit of parts comprises a containment containing a dosage form comprising the SGLT2 inhibitor and at least one pharmaceutically acceptable carrier.

A further aspect of the present invention is a manufacture comprising the pharmaceutical composition being present as separate dosage forms according to the present invention and a label or package insert comprising instructions that the separate dosage forms are to be administered in combination or alternation.

According to a first embodiment a manufacture comprises (a) a pharmaceutical composition comprising a SGLT2 inhibitor according to the present invention and (b) a label or package insert which comprises instructions that the medicament is to be administered.

The desired dose of the pharmaceutical composition according to this invention may conveniently be presented in a once daily or as divided dose administered at appropriate intervals, for example as two, three or more doses per day.

The pharmaceutical composition may be formulated for oral, rectal, nasal, topical (including buccal and sublingual), transdermal, vaginal or parenteral (including intramuscular, sub-cutaneous and intravenous) administration in liquid or solid form or in a form suitable for administration by inhalation or insufflation. Oral administration is preferred. The formulations may, where appropriate, be conveniently presented in discrete dosage units and may be prepared by any of the methods well known in the art of pharmacy. All methods include the step of bringing into association the active ingredient with one or more pharmaceutically acceptable carriers, like liquid carriers or finely divided solid carriers or both, and then, if necessary, shaping the product into the desired formulation.

The pharmaceutical composition may be formulated in the form of tablets, granules, fine granules, powders, capsules, caplets, soft capsules, pills, oral solutions, syrups, dry syrups, chewable tablets, troches, effervescent tablets, drops, suspension, fast dissolving tablets, oral fast-dispersing tablets, etc.

The pharmaceutical composition and the dosage forms preferably comprises one or more pharmaceutical acceptable carriers which must be “acceptable” in the sense of being compatible with the other ingredients of the formulation and not deleterious to the recipient thereof. Examples of pharmaceutically acceptable carriers are known to the one skilled in the art.

Pharmaceutical compositions suitable for oral administration may conveniently be presented as discrete units such as capsules, including soft gelatin capsules, cachets or tablets each containing a predetermined amount of the active ingredient; as a powder or granules; as a solution, a suspension or as an emulsion, for example as syrups, elixirs or self-emulsifying delivery systems (SEDDS). The active ingredients may also be presented as a bolus, electuary or paste. Tablets and capsules for oral administration may contain conventional excipients such as binding agents, fillers, lubricants, disintegrants, or wetting agents. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives.

The pharmaceutical composition according to the invention may also be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilisation from solution, for constitution with a suitable vehicle, e.g. sterile, pyrogen-free water, before use.

Pharmaceutical compositions suitable for rectal administration wherein the carrier is a solid are most preferably presented as unit dose suppositories. Suitable carriers include cocoa butter and other materials commonly used in the art, and the suppositories may be conveniently formed by admixture of the active compound(s) with the softened or melted carrier(s) followed by chilling and shaping in moulds.

The pharmaceutical compositions and methods according to this invention show advantageous effects in the treatment and prevention of those diseases and conditions as described hereinbefore. Advantageous effects may be seen for example with respect to efficacy, dosage strength, dosage frequency, pharmacodynamic properties, pharmacokinetic properties, fewer adverse effects, convenience, compliance, etc.

Methods for the manufacture of SGLT2 inhibitors according to this invention and of prodrugs thereof are known to the one skilled in the art. Advantageously, the compounds according to this invention can be prepared using synthetic methods as described in the literature, including patent applications as cited hereinbefore. Preferred methods of manufacture are described in the WO 2006/120208 and WO 2007/031548. With regard to empagliflozin an advantageous crystalline form is described in the international patent application WO 2006/117359 which hereby is incorporated herein in its entirety.

The active ingredients may be present in the form of a pharmaceutically acceptable salt.

Pharmaceutically acceptable salts include, without being restricted thereto, such as salts of inorganic acid like hydrochloric acid, sulfuric acid and phosphoric acid; salts of organic carboxylic acid like oxalic acid, acetic acid, citric acid, malic acid, benzoic acid, maleic acid, fumaric acid, tartaric acid, succinic acid and glutamic acid and salts of organic sulfonic acid like methanesulfonic acid and p-toluenesulfonic acid. The salts can be formed by combining the compound and an acid in the appropriate amount and ratio in a solvent and decomposer. They can be also obtained by the cation or anion exchange from the form of other salts.

The active ingredients or a pharmaceutically acceptable salt thereof may be present in the form of a solvate such as a hydrate or alcohol adduct.

Pharmaceutical compositions or combinations for use in these therapies comprising the SGLT-2 inhibitor as defined herein optionally together with one or more other active substances are also contemplated.

Further, the present invention relates to the SGLT-2 inhibitors, optionally in combination with one, two or more further active agents, each as defined herein, for use in the therapies as described herein.

Further, the present invention relates to the use of the SGLT-2 inhibitors, optionally in combination with one, two or more further active agents, each as defined herein, for preparing pharmaceutical compositions which are suitable for the treatment and/or prevention purposes of this invention.

The present invention further relates to a combination comprising a certain SGLT-2 inhibitor (particularly empagliflozin) and one or more other active substances selected from other antidiabetic substances, particularly for simultaneous, separate or sequential use in the therapies described herein.

The present invention further relates to a combination comprising a certain SGLT-2 inhibitor (particularly empagliflozin) and one or more other antidiabetics selected from the group consisting of metformin, a sulphonylurea, nateglinide, repaglinide, a thiazolidinedione, a PPAR-gamma-agonist, an alpha-glucosidase inhibitor, insulin or an insulin analogue, GLP-1 or a GLP-1 analogue and a DPP-4 inhibitor, particularly for simultaneous, separate or sequential use in the therapies described herein.

The present invention further relates to a method according to the present invention further comprising treating and/or preventing metabolic disorders, especially type 2 diabetes mellitus and/or conditions related thereto (e.g. diabetic complications) comprising the combined (e.g. simultaneous, separate or sequential) administration of an effective amount of empagliflozin and one or more other antidiabetics selected from the group consisting of metformin, a sulphonylurea, nateglinide, repaglinide, a PPAR-gamma-agonist, an alpha-glucosidase inhibitor, insulin or an insulin analogue, GLP-1 or a GLP-1 analogue and a DPP-4 inhibitor, to the patient (particularly human patient) in need thereof, such as e.g. a patient as described herein.

The present invention further relates to therapies or therapeutic methods described herein, further comprising treating and/or preventing metabolic disorders, especially type 2 diabetes mellitus and/or conditions related thereto (e.g. diabetic complications), comprising administering a therapeutically effective amount of empagliflozin and, optionally, one or more other therapeutic agents, such as e.g. antidiabetics selected from the group consisting of metformin, a sulphonylurea, nateglinide, repaglinide, a PPAR-gamma-agonist, an alpha-glucosidase inhibitor, insulin or an insulin analogue, GLP-1 or a GLP-1 analogue and a DPP-4 inhibitor, to the patient (particularly human patient) in need thereof, such as e.g. a patient as described herein.

Unless otherwise noted, combination therapy may refer to first line, second line or third line therapy, or initial or add-on combination therapy or replacement therapy.

The present invention further relates to a certain SGLT-2 inhibitor as defined herein, preferably empagliflozin, in combination with metformin, for use in the therapies described herein.

Metformin is usually given in doses varying from about 500 mg to 2000 mg up to 2500 mg per day using various dosing regimens from about 100 mg to 500 mg or 200 mg to 850 mg (1-3 times a day), or about 300 mg to 1000 mg once or twice a day, or delayed-release metformin in doses of about 100 mg to 1000 mg or preferably 500 mg to 1000 mg once or twice a day or about 500 mg to 2000 mg once a day. Particular dosage strengths may be 250, 500, 625, 750, 850 and 1000 mg of metformin hydrochloride.

For children 10 to 16 years of age, the recommended starting dose of metformin is 500 mg given once daily. If this dose fails to produce adequate results, the dose may be increased to 500 mg twice daily. Further increases may be made in increments of 500 mg weekly to a maximum daily dose of 2000 mg, given in divided doses (e.g. 2 or 3 divided doses). Metformin may be administered with food to decrease nausea.

An example of a DPP-4 inhibitor is linagliptin, which is usually given in a dosage of 5 mg per day.

A dosage of pioglitazone is usually of about 1-10 mg, 15 mg, 30 mg, or 45 mg once a day.

Rosiglitazone is usually given in doses from 4 to 8 mg once (or divided twice) a day (typical dosage strengths are 2, 4 and 8 mg).

Glibenclamide (glyburide) is usually given in doses from 2.5-5 to 20 mg once (or divided twice) a day (typical dosage strengths are 1.25, 2.5 and 5 mg), or micronized glibenclamide in doses from 0.75-3 to 12 mg once (or divided twice) a day (typical dosage strengths are 1.5, 3, 4.5 and 6 mg).

Glipizide is usually given in doses from 2.5 to 10-20 mg once (or up to 40 mg divided twice) a day (typical dosage strengths are 5 and 10 mg), or extended-release glibenclamide in doses from 5 to 10 mg (up to 20 mg) once a day (typical dosage strengths are 2.5, 5 and 10 mg).

Glimepiride is usually given in doses from 1-2 to 4 mg (up to 8 mg) once a day (typical dosage strengths are 1, 2 and 4 mg).

The non-sulphonylurea insulin secretagogue nateglinide is usually given in doses from 60 to 120 mg with meals (up to 360 mg/day, typical dosage strengths are 60 and 120 mg); repaglinide is usually given in doses from 0.5 to 4 mg with meals (up to 16 mg/day, typical dosage strengths are 0.5, 1 and 2 mg). A dual combination of repaglinide/metformin is available in dosage strengths of 1/500 and 2/850 mg.

In one aspect of the present invention, the one or more other therapeutic substances are RAAS inhibitors (Renin-Angiotensin-Aldosterone System). In one aspect of the present invention, the one or more other therapeutic substances is a direct renin inhibitor, an

Angiotensin-Converting Enzyme (ACE) inhibitor and/or an angiotensin II receptor blocker (ARB).

In one aspect, a SGLT-2 inhibitor, in particular empagliflozin is used in a method according to the present invention in addition to a RAAS inhibitor, in particular a direct renin inhibitor, an

Angiotensin-Converting Enzyme (ACE) inhibitor and/or an angiotensin II receptor blocker (ARB).

In another embodiment of the present invention, certain SGLT-2 inhibitors, in particular empagliflozin, are useful in the therapy of a patient with chronic kidney disease and albuminuria despite therapy with a direct renin inhibitor, an angiotensin-converting enzyme (ACE) inhibitor and/or an angiotensin II receptor blocker (ARB), in particular a patient described herein.

In another embodiment of the present invention, certain a SGLT-2 inhibitors, in particular empagliflozin, are useful in a method according to the present invention on top of direct renin inhibitor therapy, angiotensin-converting enzyme (ACE) inhibitor therapy and/or angiotensin II receptor blockade (ARB) therapy.

Accordingly, in a further embodiment, the present invention relates to a certain SGLT-2 inhibitor, in particular empagliflozin, in combination with a RAAS inhibitor, for example a direct renin inhibitor, an Angiotensin-Converting Enzyme (ACE) inhibitor and/or an angiotensin II receptor blocker (ARB), for use in the therapy as described herein, for example in a patient as described herein.

In one aspect, the present invention relates to certain SGLT-2 inhibitors, for example empagliflozin, in combination with a RAAS inhibitor, for example a direct renin inhibitor, an Angiotensin-Converting Enzyme (ACE) inhibitor and/or an angiotensin II receptor blocker (ARB), for use in treating, preventing, protecting against, reducing the risk of, delaying the occurrence of and/or delaying the progression of chronic kidney disease in patients, for example patients with prediabetes, type 1 or type 2 diabetes mellitus.

In a further aspect, the present invention relates to certain SGLT-2 inhibitors, for example empagliflozin, in combination with a RAAS inhibitor, for example a direct renin inhibitor, an Angiotensin-Converting Enzyme (ACE) inhibitor and/or an angiotensin II receptor blocker

(ARB), for use in treating, preventing, protecting against or delaying new onset of albuminuria in patients.

In a further aspect, the present invention relates to certain SGLT-2 inhibitors, for example empagliflozin, in combination with a RAAS inhibitor, for example a direct renin inhibitor, an

Angiotensin-Converting Enzyme (ACE) inhibitor and/or an angiotensin II receptor blocker (ARB), for use in treating, preventing, protecting against or delaying the progression from no albuminuria to micro- or macroalbuminuria in a patient at risk for renal disease.

In a further aspect, the present invention relates to certain SGLT-2 inhibitors, for example empagliflozin, in combination with a RAAS inhibitor, for example a direct renin inhibitor, an Angiotensin-Converting Enzyme (ACE) inhibitor and/or an angiotensin II receptor blocker (ARB), for use in treating, preventing, protecting against or delaying the progression from microalbuminuria to macroalbuminuria in a patient with chronic kidney disease.

In a further aspect, the present invention relates to certain SGLT-2 inhibitors, for example empagliflozin, in combination with a RAAS inhibitor, for example a direct renin inhibitor, an Angiotensin-Converting Enzyme (ACE) inhibitor and/or an angiotensin II receptor blocker (ARB), for use in treating, preventing, protecting against delaying the progression of chronic kidney disease in a patient with chronic kidney disease.

In a further aspect, the present invention relates to certain SGLT-2 inhibitors, for example empagliflozin, in combination with a RAAS inhibitor, for example a direct renin inhibitor, an Angiotensin-Converting Enzyme (ACE) inhibitor and/or an angiotensin II receptor blocker (ARB), for use in treating, preventing, protecting against or delaying the occurrence of:
new onset of albuminuria,
doubling of serum creatinine level accompanied by an eGFR (based on modification of diet in renal disease (MDRD) formula) ≤45 mL/min/1.73 m²,
need for continuous renal replacement therapy, or
death due to renal disease.

In one aspect, a patient according of the present invention is a patient with prediabetes, type 1 or type 2 diabetes mellitus. In one aspect, a patient according to the present invention is a patient is a patient at risk for renal disease. In one aspect, a patient according to the present invention is a patient with or at risk of a cardiovascular disease. In one aspect, a patient according to the present invention is a patient with prediabetes, type 1 or type 2 diabetes mellitus and with or at risk of a cardiovascular disease.

Examples of Angiotensin-Converting Enzyme (ACE) inhibitors are Benazepril, Captopril, ramipril, lisinopril, Moexipril, cilazapril, quinapril, captopril, enalapril, benazepril, perindopril, fosinopril and trandolapril; the dosage(s) of some of these medications are for example shown below:
Benazepril (Lotensin), 5 mg, 10 mg, 20 mg, and 40 mg for oral administration
Captopril (Capoten), 12.5 mg, 25 mg, 50 mg, and 100 mg as scored tablets for oral administration
Enalapril (Vasotec), 2.5 mg, 5 mg, 10 mg, and 20 mg tablets for oral administration
Fosinopril (Monopril), for oral administration as 10 mg, 20 mg, and 40 mg tablets
Lisinopril (Prinivil, Zestril), 5 mg, 10 mg, and 20 mg tablets for oral administration
Moexipril (Univasc), 7.5 mg and 15 mg for oral administration
Perindopril (Aceon), 2 mg, 4 mg and 8 mg strengths for oral administration
Quinapril (Accupril), 5 mg, 10 mg, 20 mg, or 40 mg of quinapril for oral administration
Ramipril (Altace), 1.25 mg, 2.5 mg, 5, mg, 10 mg
Trandolapril (Mavik) , 1 mg, 2 mg, or 4 mg of trandolapril for oral administration

Examples of angiotensin II receptor blockers (ARBs) are telmisartan, candesartan, valsartan, losartan, irbesartan, olmesartan, azilsartan and eprosartan; the dosage(s) of some of these medications are for example shown below:
Candesartan (Atacand), 4 mg, 8 mg, 16 mg, or 32 mg of candesartan cilexetil
Eprosartan (Teveten), 400 mg or 600 mg
Irbesartan (Avapro), 75 mg, 150mg, or 300 mg of irbesartan.
Losartan (Cozaar), 25 mg, 50 mg or 100 mg of losartan potassium
Telmisartan (Micardis) , 40 mg/12.5 mg, 80 mg/12.5 mg, and 80 mg/25 mg telmisartan and hydrochlorothiazide
Valsartan (Diovan) , 40 mg, 80 mg, 160 mg or 320 mg of valsartan

A dosage of telmisartan is usually from 20 mg to 320 mg or 40 mg to 160 mg per day.

An example of a direct renin inhibitor is aliskiren (Tekturna). A dosage of aliskiren may be 150 mg or 300 mg per day.

Within this invention it is to be understood that the combinations, compositions or combined uses according to this invention may envisage the simultaneous, sequential or separate administration of the active components or ingredients.

In this context, “combination” or “combined” within the meaning of this invention may include, without being limited, fixed and non-fixed (e.g. free) forms (including kits) and uses, such as e.g. the simultaneous, sequential or separate use of the components or ingredients.

The combined administration of this invention may take place by administering the active components or ingredients together, such as e.g. by administering them simultaneously in one single or in two separate formulations or dosage forms. Alternatively, the administration may take place by administering the active components or ingredients sequentially, such as e.g. successively in two separate formulations or dosage forms.

For the combination therapy of this invention the active components or ingredients may be administered separately (which implies that they are formulated separately) or formulated altogether (which implies that they are formulated in the same preparation or in the same dosage form). Hence, the administration of one element of the combination of the present invention may be prior to, concurrent to, or subsequent to the administration of the other element of the combination.

In a further aspect, the present invention provides a pharmaceutical composition comprising a SGLT-2 inhibitor, for example empagliflozin, in combination with a RAAS inhibitor, for example a direct renin inhibitor, an Angiotensin-Converting Enzyme (ACE) inhibitor and/or an angiotensin II receptor blocker (ARB), for example as described herein.

Further embodiments, features and advantages of the present invention may become apparent from the following examples. The following examples serve to illustrate, by way of example, the principles of the invention without restricting it.

### EXAMPLES

### Example 1: Empagliflozin in Patients With Type 2 Diabetes Mellitus (T2DM) and Renal Impairment (RI)

A Phase III trial investigated the efficacy and safety of empagliflozin (EMPA) as add-on to existing therapy for 52 weeks in patients with T2DM and RI. Patients with mild RI (eGFR [MDRD equation] ≥60 to <90 mL/min/1.73 m²) received EMPA 10 or 25 mg qd or placebo (PBO). Patients with moderate RI (eGFR ≥30 to <60 mL/min/1.73 m²) received EMPA 25 mg qd or PBO. Patients with severe RI (eGFR ≥15 to <30 mL/min/1.73 m²) received EMPA 25 mg qd or

PBO.

In patients with type 2 diabetes and mild renal impairment, treatment with empagliflozin 10 and 25 mg at week 52 resulted in a small decrease in eGFR. However, mean eGFR increased to a value slightly above baseline at the 3-week follow up visit in the empagliflozin treatment groups; in contrast, in the placebo group, mean eGFR further decreased (Table 1A).

TABLE 1A
Descriptive statistics for eGFR over time
in patients with mild renal impairment

| | Placebo | | Empa 10 mg | | Empa 25 mg | |
|---|---|---|---|---|---|---|
| Number of patients | 32 | (100.0) | 41 | (100.0) | 38 | (100.0) |
| N (%) | | | | | | |
| Baseline eGFR | | | | | | |
| N* (%) | 32 | (100.0) | 41 | (100.0) | 38 | (100.0) |
| Mean (SD) | 72.24 | (12.68) | 68.42 | (8.23) | 72.01 | (10.84) |
| [mL/min/1.73 m²] | | | | | | |
| End-of treatment | | | | | | |
| eGFR | | | | | | |
| N* (%) | 32 | (100.0) | 38 | (92.7) | 37 | (97.4) |
| Mean (SD) | 70.34 | (11.42) | 68.07 | (11.36) | 66.25 | (13.00) |
| [mL/min/1.73 m²] | | | | | | |
| Mean change from | −1.89 | (11.14) | −0.76 | (9.42) | −5.67 | (10.37) |
| baseline (SD) | | | | | | |
| [mL/min/1.73 m²] | | | | | | |
| Follow-up eGFR | | | | | | |
| N* (%) | 30 | (93.8) | 38 | (92.7) | 37 | (97.4) |
| Mean (SD) | 68.20 | (11.16) | 69.84 | (11.29) | 73.38 | (13.67) |
| [mL/min/1.73 m²] | | | | | | |
| Mean change from | −3.84 | (11.63) | 2.06 | (8.91) | 1.28 | (8.89) |
| baseline (SD) | | | | | | |
| [mL/min/1.73 m²] | | | | | | |

In patients with type 2 diabetes and moderate renal impairment, treatment with empagliflozin 25 mg at week 52 resulted a small decrease in eGFR while no change was seen for the placebo group. However, at the 3-week follow-up visit mean eGFR increased to a value slightly above baseline in the empagliflozin treatment group (Table 1B). Similar results were seen in patients with CKD 3A and B.

TABLE 1B
Descriptive statistics for eGFR over time
in patients with moderate renal impairment

| | Placebo | | Empa 25 mg | |
|---|---|---|---|---|
| Number of patients N (%) | 104 | (100.0) | 105 | (100.0) |
| Baseline eGFR | | | | |
| N* (%) | 104 | (100.0) | 105 | (100.0) |
| Mean (SD) [mL/min/1.73 m²] | 43.35 | (10.39) | 43.84 | (8.70) |
| End-of treatment eGFR | | | | |
| N* (%) | 102 | (98.1) | 101 | (96.2) |
| Mean (SD) [mL/min/1.73 m²] | 43.70 | (11.08) | 40.58 | (10.26) |
| Mean change from baseline (SD) | 0.04 | (7.16) | −3.55 | (6.63) |
| [mL/min/1.73 m²] | | | | |
| Follow-up eGFR | | | | |
| N* (%) | 98 | (94.2) | 103 | (98.1) |
| Mean (SD) [mL/min/1.73 m²] | 42.99 | (12.67) | 45.39 | (11.31) |
| Mean change from baseline (SD) | 0.16 | (9.14) | 1.48 | (6.70) |
| [mL/min/1.73 m²] | | | | |

In patients with type 2 diabetes and severe renal impairment, treatment with empagliflozin 25 mg at week 52 resulted in a small decrease in eGFR. However, at the 3-week follow-up visit mean eGFR increased to a value slightly below baseline in the empagliflozin treatment group (Table 1C).

TABLE 1C
Descriptive statistics for eGFR over time
in patients with severe renal impairment

| | Placebo | | Empa 25 mg | |
|---|---|---|---|---|
| Number of patients N (%) | 18 | (100.0) | 21 | (100.0) |
| Baseline eGFR | | | | |
| N* (%) | 18 | (100.0) | 21 | (100.0) |
| Mean (SD) [mL/min/1.73 m²] | 22.90 | (3.44) | 24.22 | (3.99) |
| End-of treatment eGFR | | | | |
| N* (%) | 17 | (94.4) | 21 | (100.0) |
| Mean (SD) [mL/min/1.73 m²] | 21.80 | (6.36) | 20.23 | (5.86) |
| Mean change from baseline (SD) | −1.17 | (5.82) | −3.98 | (5.80) |
| [mL/min/1.73 m²] | | | | |
| Follow-up eGFR | | | | |
| N* (%) | 18 | (100.0) | 21 | (100.0) |
| Mean (SD) [mL/min/1.73 m²] | 21.42 | (6.58) | 23.63 | (7.40) |
| Mean change from baseline (SD) | −1.48 | (6.03) | −0.59 | (6.76) |
| [mL/min/1.73 m²] | | | | |

### Example 2: Empagliflozin in Hypertensive Patients With Type 2 Diabetes Mellitus (T2DM)

A Phase III trial investigated the efficacy and safety of empagliflozin (EMPA) administered 10 orally, once daily over 12 weeks in hypertensive patients with T2DM (EMPA 10 or 25 mg, placebo (PBO)). Patients with a systolic blood pressure (SBP) of 130 to 159 mmHg and a diastolic blood pressure (DSP) of 80 to 99 mmHg were included in the trial.

Treatment with empagliflozin 10 and 25 mg at week 12 resulted in a small decrease in eGFR. However, mean eGFR increased to a value slightly above baseline at the 2-week follow up visit in the empagliflozin treatment groups; in contrast, in the placebo group, mean eGFR remained slightly below baseline (Table 2).

TABLE 2
Descriptive statistics for eGFR (MDRD) over time

| | Placebo | | Empa 10 mg | | Empa 25 mg | |
|---|---|---|---|---|---|---|
| Baseline eGFR | | | | | | |
| N* (%) | 238 | (100) | 241 | (100) | 244 | (100) |
| Mean (SD) | 84.47 | (17.06) | 83.01 | (16.43) | 83.97 | (17.85) |
| [mL/min/1.73 m²] | | | | | | |
| Last value on | | | | | | |
| treatment eGFR | | | | | | |
| N* (%) | 237 | (99.6) | 238 | (98.8) | 240 | (98.4) |
| Mean (SD) | 84.16 | (17.95) | 82.70 | (17.11) | 81.24 | (17.61) |
| [mL/min/1.73 m²] | | | | | | |
| Mean change from | −0.27 | (9.18) | −0.20 | (8.99) | −2.60 | (9.98) |
| baseline (SD) | | | | | | |
| [mL/min/1.73 m²] | | | | | | |
| Follow-up eGFR | | | | | | |
| N* (%) | 236 | (99.2) | 238 | (98.8) | 243 | (99.6) |
| Mean (SD) | 83.52 | (17.37) | 86.25 | (17.06) | 86.60 | (18.24) |
| [mL/min/1.73 m²] | | | | | | |
| Mean change from | −0.82 | (9.62) | 3.06 | (10.05) | 2.75 | (9.71) |
| baseline (SD) | | | | | | |
| [mL/min/1.73 m²] | | | | | | |
| N* (%) | 236 | (99.2) | 236 | (97.9) | 239 | (98.0) |
| Mean change from | −0.52 | (9.39) | 3.32 | (9.75) | 5.54 | (9.44) |
| last value on | | | | | | |
| treatment (SD) | | | | | | |
| [mL/min/1.73 m²] | | | | | | |

*Patients with values at this time point

### Example 3: Empagliflozin in Patients With Type 2 Diabetes Mellitus (T2DM) Receiving Treatment With Basal Insulin

A Phase IIb trial investigated the efficacy and safety of empagliflozin (EMPA 10 or 25 mg, placebo (PBO)) administered orally, once daily over 78 weeks in patients with T2DM receiving treatment with basal insulin (glargine, detemir, or NPH insulin only).

Treatment with empagliflozin 10 and 25 mg resulted in a small decrease in eGFR. However, mean eGFR increased to a value slightly below baseline at the 4-week follow up visit in the empagliflozin treatment groups; in contrast, in the placebo group, mean eGFR further slightly decreased (Table 3).

TABLE 3
Descriptive statistics for eGFR (MDRD) over time

| | Placebo | | Empa 10 mg | | Empa 25 mg | |
|---|---|---|---|---|---|---|
| Number of patients, | 170 | (100.0) | 169 | (100.0) | 155 | (100.0) |
| N (%) | | | | | | |
| Baseline eGFR | | | | | | |
| N¹ (%) | 170 | (100.0) | 169 | (100.0) | 155 | (100.0) |
| Mean (SD) | 83.89 | (22.73) | 85.01 | (23.63) | 82.88 | (25.46) |
| Week 18 eGFR | | | | | | |
| N¹ (%) | 134 | (78.8) | 133 | (78.7) | 113 | (72.9) |
| Mean (SD) | 80.07 | (20.15) | 80.37 | (23.03) | 79.11 | (21.63) |
| Mean change from | −4.12 | (12.27) | −4.98 | (11.40) | −3.48 | (10.10) |
| baseline (SD) | | | | | | |
| Week 54 eGFR | | | | | | |
| N¹ (%) | 106 | (62.4) | 106 | (62.7) | 97 | (62.6) |
| Mean (SD) | 78.54 | (21.06) | 82.55 | (23.60) | 77.12 | (23.59) |
| Mean change from | −4.88 | (10.85) | −5.68 | (14.36) | −4.76 | (11.05) |
| baseline (SD) | | | | | | |
| Week 78 eGFR | | | | | | |
| N¹ (%) | 102 | (60.0) | 100 | (59.2) | 86 | (55.5) |
| Mean (SD) | 78.52 | (21.11) | 81.86 | (24.17) | 77.21 | (20.68) |
| Mean change from | −5.27 | (12.04) | −5.52 | (11.08) | −5.64 | (10.20) |
| baseline (SD) | | | | | | |
| eGFR at follow-up | | | | | | |
| N¹ (%) | 112 | | 118 | | 113 | |
| Mean (SD) | 78.36 | (21.39) | 83.74 | (21.69) | 81.35 | (21.78) |
| Mean change from | −6.66 | (12.06) | −1.88 | (13.02) | −0.79 | (12.00) |
| baseline (SD) | | | | | | |

¹Percent of patient in population with values at this time point
eGFR = estimated glomerular filtration rate; MDRD = Modification of diet in renal disease; SD = standard deviation

### Example 4: Empagliflozin in Patients With Type 2 Diabetes Mellitus (T2DM) and Microalbuminuria and Macroalbuminuria

In a dedicated 52 week renal impairment study, patients were categorised based on their urine albumin/creatinine ratio (UACR) values at baseline, and 3 categories of patients were distinguished: patients with normal urine albumin/creatinine ratio values (<30 mg/g), patients with microalbuminuria (30 to <300 mg/g) and patients with macroalbuminurea (≥300 mg/g). Differences in mean changes from baseline between the placebo and the empagliflozin treatment groups were noted for patients with microalbuminuria or macroalbuminuria at baseline. For patients with microalbuminuria, mean urine albumin/creatinine ratio values increased with placebo treatment, remained nearly unchanged with empagliflozin 10 mg treatment, and decreased with empagliflozin 25 mg treatment. In patients with macroalbuminuria at baseline, a decrease in mean urine albumin/creatinine ratio was noted only in the empagliflozin groups; (Table 4A).

TABLE 4A
Urine albumin-to-creatinine ratio (mg/g) by baseline urine
albumin-to-creatinine ratio at week 52 in normal patients
and patients with microalbuminuria and macroalbuminuria

| | Change from baseline | | | | | | | | |
|---|---|---|---|---|---|---|---|---|---|
| | Normal | | | Microalbuminuria | | | Macroalbuminuria | | |
| | N | Mean | SD | N | Mean | SD | N | Mean | SD |
| Placebo | 134 | 8.2 | 20.8 | 90 | 106.1 | 412.9 | 50 | 3.4 | 2050.5 |
| Empagliflozin | 59 | 4.0 | 15.3 | 17 | 7.4 | 137.3 | 9 | −716.3 | 1273.6 |
| 10 mg | | | | | | | | | |
| Empagliflozin | 141 | 6.0 | 31.7 | 72 | −39.4 | 93.8 | 61 | −799.9 | 1543.7 |
| 25 mg | | | | | | | | | |

Shifts between UACR categories at baseline and the end of treatment were noted in the randomised treatment groups (Table 4B). A higher frequency of patients in the empagliflozin treatment groups shifted from macro- or microalbuminura at baseline towards normal values and from macro- to microalbuminuria at the end of treatment. In addition, a higher proportion of patients in the placebo group shifted from normal values at baseline towards microalbuminuria at the end of treatment.

TABLE 4B
Frequency of patients [N (%)] with shifts in urine
albumin-to-creatinine ratio (mg/g) at Week 52 relative
to urine albumin-to-creatinine ratio categories at baseline

| | | Last value on-treatment | | | | | |
|---|---|---|---|---|---|---|---|
| | | | | Micro- | | Macro- | |
| Treat- | | Normal | | albuminuria | | albuminuria | |
| ment | Baseline | N (%) | | N (%) | | N (%) | |
| Placebo | Normal | 118 | (81.9) | 26 | (18.1) | 0 | |
| | Microalbuminuria | 22 | (21.8) | 67 | (66.3) | 12 | (11.9) |
| | Macroalbuminuria | 1 | (1.7) | 6 | (10.0) | 53 | (88.3) |
| Empa | Normal | 59 | (89.4) | 7 | (10.6) | 0 | |
| 10 mg | Microalbuminuria | 5 | (27.8) | 11 | (61.1) | 2 | (11.1) |
| | Macroalbuminuria | 1 | (11.1) | 5 | (55.6) | 3 | (33.3) |
| Empa | Normal | 135 | (89.4) | 16 | (10.6) | 0 | |
| 25 mg | Microalbuminuria | 19 | (24.1) | 57 | (72.2) | 3 | (3.8) |
| | Macroalbuminuria | 2 | (2.7) | 22 | (30.1) | 49 | (67.1) |

Categories for urine albumin-to-creatinine ratio: normal: <30 mg/g, microalbuminuria 30 to <300 mg/g, macroalbuminuria: ≥300 mg/g

### Example of Pharmaceutical Composition and Dosage Form

The following example of solid pharmaceutical compositions and dosage forms for oral administration serves to illustrate the present invention more fully without restricting it to the contents of the example. Further examples of compositions and dosage forms for oral administration, are described in WO 2010/092126. The term “active substance” denotes empagliflozin according to this invention, especially its crystalline form as described in WO 2006/117359 and WO 2011/039107.

Tablets containing 2.5 mg, 5 mg, 10
mg, 25 mg, 50 mg of active substance

| | 2.5 | 5 | 10 | 25 | 50 |
|---|---|---|---|---|---|
| | mg/per | mg/per | mg/per | mg/per | mg/per |
| Active substance | tablet | tablet | tablet | tablet | tablet |
| Wet granulation | | | | | |
| active substance | 2.5000 | 5.000 | 10.00 | 25.00 | 50.00 |
| Lactose | 40.6250 | 81.250 | 162.50 | 113.00 | 226.00 |
| Monohydrate | | | | | |
| Microcrystalline | 12.5000 | 25.000 | 50.00 | 40.00 | 80.00 |
| Cellulose | | | | | |
| Hydroxypropyl | 1.8750 | 3.750 | 7.50 | 6.00 | 12.00 |
| Cellulose | | | | | |
| Croscarmellose | 1.2500 | 2.500 | 5.00 | 4.00 | 8.00 |
| Sodium | | | | | |
| Purified Water | q.s. | q.s. | q.s. | q.s. | q.s. |
| Dry Adds | | | | | |
| Microcrystalline | 3.1250 | 6.250 | 12.50 | 10.00 | 20.00 |
| Cellulose | | | | | |
| Colloidal silicon | 0.3125 | 0.625 | 1.25 | 1.00 | 2.00 |
| dioxide | | | | | |
| Magnesium stearate | 0.3125 | 0.625 | 1.25 | 1.00 | 2.00 |
| Total core | 62.5000 | 125.000 | 250.00 | 200.00 | 400.00 |
| Film Coating | | | | | |
| Film coating | 2.5000 | 4.000 | 7.00 | 6.00 | 9.00 |
| system | | | | | |
| Purified Water | q.s. | q.s. | q.s. | q.s. | q.s. |
| Total | 65.000 | 129.000 | 257.00 | 206.00 | 409.00 |

Details regarding the manufacture of the tablets, the active pharmaceutical ingredient, the excipients and the film coating system are described in WO 2010/092126, in particular in the Examples 5 and 6, which hereby is incorporated herein in its entirety.

1. A method for delaying loss of eGFR and/or for delaying the progression to end stage renal disease/kidney failure, or renal death in a patient with chronic kidney disease, said method comprising administering empagliflozin, optionally in combination with one or more other therapeutic substances, to the patient.

2. The method according to claim 1, wherein the patient with chronic kidney disease is a patient with albuminuria.

3. The method according to claim 2, wherein the patient with chronic kidney disease is a patient with microalbuminuria or with macroalbuminuria.

4. The method according to claim 1, wherein the patient is a patient with prediabetes, type 1 or type 2 diabetes mellitus.

5. The method according to claim 1, wherein the patient has or is at risk of a cardiovascular disease.

6. The method according to claim 1, wherein the patient is a patient with prediabetes, type 1 or type 2 diabetes mellitus and with or at risk of a cardiovascular disease.

7. The method according to claim 1, wherein the patient is a patient with stage 1 chronic kidney disease, stage 2 chronic kidney disease, stage 3 chronic kidney disease, stage 4 chronic kidney disease, or stage 5 chronic kidney disease.

8. The method according to claim 7, wherein the patient is a patient with stage 2 chronic kidney disease, stage 3 chronic kidney disease or stage 4 chronic kidney disease.

9. The method according to claim 1, wherein the patient is a patient with one or more cardiovascular risk factors selected from A), B), C) and D):
A) previous or existing vascular disease selected from myocardial infarction, coronary artery disease, percutaneous coronary intervention, coronary artery by-pass grafting, ischemic or hemorrhagic stroke, congestive heart failure, and peripheral occlusive arterial disease,
B) advanced age >/=60-70 years, and
C) one or more cardiovascular risk factors selected from
advanced type 1 or type 2 diabetes mellitus >10 years duration,
hypertension,
current daily cigarette smoking,
dyslipidemia,
obesity,
age >/=40,
metabolic syndrome, hyperinsulinemia or insulin resistance, and
hyperuricemia, erectile dysfunction, polycystic ovary syndrome, sleep apnea, or family history of vascular disease or cardiomyopathy in first-degree relative;
D) one or more of the following:
confirmed history of myocardial infarction,
unstable angina with documented multivessel coronary disease or positive stress test,
multivessel Percutaneous Coronary Intervention,
multivessel Coronary Artery By-pass Grafting (CABG),
history of ischemic or hemorrhagic stroke,
peripheral occlusive arterial disease.

10. The method according to claim 1, wherein the one or more other therapeutic substances are selected from other antidiabetic substances.

11. The method according to claim 1, comprising administering empagliflozin in combination with metformin, with linagliptin or with metformin and linagliptin.

12. The method according to claim 1, wherein the one or more other therapeutic substances is a RAAS inhibitor.

13. The method according to claim 1, wherein the one or more other therapeutic substances is a direct renin inhibitor, an Angiotensin-Converting Enzyme (ACE) inhibitor and/or an angiotensin II receptor blocker (ARB).

14. The method according to claim 1, comprising administering empagliflozin in combination with a RAAS inhibitor.

15. The method according to claim 1, comprising administering empagliflozin in combination with a direct renin inhibitor, an Angiotensin-Converting Enzyme (ACE) inhibitor and/or an angiotensin II receptor blocker (ARB).

16. The method according to claim 1, wherein empagliflozin is administered orally in a total daily amount of 10 mg or 25 mg.

17. The method according to claim 1, wherein empagliflozin is administered as a pharmaceutical composition comprising 10 mg or 25 mg of empagliflozin.
